(19) United States
(12) Patent Application Publication
(10) Pub. No.: US 2010/0087326 A1
(43) Pub. Date: Apr. 8, 2010

(54) HETEROCYLIC COMPOUNDS CONTAINING THE MORPHOLINE NUCLEUS THEIR PREPARATION AND USE

(75) Inventors: Antonio Guarna, Milano (IT); Andrea Trabocchi, Firenze (IT); Gloria Menchi, Sesto Florentino (IT); Claudia Lalli, Firenze (IT); Filippo Sladojevich, S. Miniato (IT); Nicoletta Cini, Firenze (IT)

Correspondence Address: SQUIRE, SANDERS & DEMPSEY L.L.P., 1 MARITIME PLAZA, SUITE 300, SAN FRANCISCO, CA, 94111, US

(73) Assignee: Minerva Patent SA

(21) Appl. No.: 12/596,822

(22) PCT Filed: Apr. 18, 2008
(86) PCT No.: PCT/EP08/54750

§ 371 Date: Oct. 20, 2009

(30) Foreign Application Priority Data
Apr. 20, 2007 (IT) FI2007A000097

(51) Int. Cl. C40B 30/00; C07D 498/04; C40B 40/04

(52) U.S. Cl. 506/7; 544/105; 506/15; 540/497

(57) ABSTRACT

Herein are described heterocyclic compounds containing the morpholine nucleus prepared from amino acids, their preparation and use for therapeutic applications.

### FIELD OF INVENTION

The present invention relates to heterocyclic compounds, specifically comprising the morpholine nucleus.

### STATE OF ART

The creation of new molecules useful for therapeutic applications is necessary for the screening of large compound libraries, in order to identify molecular structures to be successively selected as new lead compounds with respect to biological targets. Thus, rapid and efficient synthetic methods are needed for producing libraries having a huge number of molecules endowed with high molecular diversity. During last decade, drug discovery focused on the generation of combinatorial libraries of ad-hoc designed molecules, especially taking advantage of solid-phase synthetic techniques, as demonstrated by the high number of papers and patents in the field. Variably substituted heterocyclic compounds and functionalizable with reactive groups for immobilization on solid supports are very useful for this new kind of synthetic strategy. Moreover, access to stereoselective processes to obtain enantiopure compounds is desirable in a chemical library useful for drug discovery. Unfortunately, most common synthetic methods in combinatorial chemistry are based on the generation of libraries through simple functionalization of a given molecule, often of cyclic or polycyclic nature, thus limiting the achievement of molecules carriers of high molecular diversity within the same synthetic process. The recently introduced concept of Diversity-Oriented Synthesis (Schreiber, S. L. Science 2000, 287, 1964-1968), has been placed as a new paradigm for the improvement of molecular diversity in the same synthetic process, which combines the generation of a densely functionalized precursor with further synthetic elaborations, in order to obtain a large array both of diverse cyclic molecules and variably decorated with functional groups. In the field of medicinal chemistry the synthesis and applications of cyclic amino acids has attracted considerable interest, particularly in the field of peptidomimetics (Gante, J. Angew. Chem. Int. Ed. Engl. 1994, 33, 1699-1720). Secondary cyclic amino acids have been extensively used in biomedical research, and their insertion in biologically active peptides has been documented in the course of the years. In particular, the morpholine ring is found in numerous bioactive molecules, such as inhibitors of TACE (Levin, J. I.; Chen, J. M.; Laakso, L. M.; Du, M.; Du, X.; Venkatesan, A. M.; Sandanayaka, V.; Zask, A.; Xu, J.; Xu, W.; Zhang, Y.; Skotnicki, J. S. Bioorg. Med. Chem. Lett. 2005, 15, 4345-4349), of MMP and TNF, and within the structure of the potent VLA-4 antagonist (Chiba, J.; Machinaga, N.; Takashi, T.; Ejima, A.; Takayama, G.; Yokoyama, M.; Nakayama, A.; Baldwin, J. J.; McDonald, E.; Saionz, K. W.; Swanson, R.; Hussain, Z.; Wong, A. Bioorg. Med. Chem. Lett. 2005, 15, 41-45). Moreover, morpholine has been successfully inserted in the heterocyclic structure of tricyclic benzodiazepines (Matthews, J. M.; Dyatkin, A. B.; Evangelisto, M.; Gauthier, D. A.; Hecker, L. R.; Hoekstra, W. J.; Liu, F.; Poulter, B. L.; Sorgi, K. L.; Maryanoff, B. E. Tetrahedron: Asymmetry 2004, 15, 1259-1267), of 6-methylidene-penem as β-lactamase inhibitors (Venkatesan, A. M.; Agarwal, A.; Abe, T.; Ushirogochi, H.; Yamamura, I.; Ado, M.; Tsuyoshi, T.; Dos Santos, O.; Gu, Y.; Sum, F.-W.; Li, Z.; Francisco, G.; Lin, Y.-I.; Petersen, P. J.; Yang, Y.; Kumagai, T.; Weiss, W. J.; Shlaes, D. M.; Knox, J. R.; Mansour, T. S. J. Med. Chem. 2006, 49, 4623-4637) of β-carbolines as IKK-2 inhibitors, of 6,8-fused bicyclic peptidomimetics as interleukin-1β converting enzyme inhibitors (O′Neil, S. V.; Wang, Y.; Laufersweiler, M. C.; Oppong, K. A.; Soper, D. L.; Wos, J. A.; Ellis, C. D.; Baize, M. W.; Bosch, G. K.; Fancher, A. N.; Lu, W.; Suchanek, M. K.; Wang, R. L.; De, B.; Demuth, Jr., T. P. Bioorg. Med. Chem. Lett. 2005, 15, 5434-5438), and in the structure of benzoxazepines as stimulators of AMPA receptor, which demonstrates the high interest in the biomedical field towards this heterocycle and the molecules containing it.

All the aforementioned features can be included in the compounds of general formula (I), which can be achieved from a two steps synthetic process using precursors easily obtainable as enantiopure compounds. Such new type of compounds, cyclic or bicyclic in structure, can be successively functionalized in different positions and transformed in other compounds containing the morpholine ring through subsequent reactions as known in the literature, thus functioning as core structure for the generation of a wide array of new compounds with high level of molecular diversity.

### DETAILED DESCRIPTION OF THE INVENTION

The present invention allows to solve the problems as above described thank to the cyclic compounds of general formula (I):

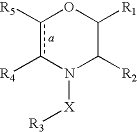

wherein:
a is a single or double bond;
X is chosen in the group consisting of “bond”, CO, SO₂, CS;
R₁ is chosen in the group consisting of H, C₁₋₈alkyl, C₂₋₈alkenyl, C₂₋₈alkynyl, C₁₋₈cycloalkyl, aryl, heterocycle, arylC₁₋₈alkyl; heterocycloC₁₋₈alkyl; —CH₂OR, RO—C₁₋₈alkyl, —CH₂NRR′, RR′N—C₁₋₈alkyl, RR′N-aryl, RO-aryl, R(O)C-aryl, RO(O)C-aryl, RR′N(O)C-aryl;
R₂ is chosen in the group consisting of α-amino acid side chain, —CO₂alkyl, —CH₂Oalkyl, —CH₂Oaryl, —CH₂OPg, —C(O)NRR′, —C(O)NHR′;
R₁ and R₂ can form a cycle;
R₃ is chosen in the group consisting of H, C₁₋₈alkyl, C₂₋₈alkenyl, C₂₋₈alkynyl, cycloalkyl, aryl, heterocicle, arylC₁₋₈alkyl; heterocycloC₁₋₈alkyl; RR′N—C₁₋₈alkyl, RR′N-aryl, RO-aryl, R(O)C-aryl, RO(O)C-aryl, RR′N(O)C-aryl, —CH(aryl)CO₂R, —CH(hetocycle)CO₂R, —CH(alkenyl)CO₂R, when X is bond;
   is chosen in the group consisting of C₁₋₈alkyl, C₂₋₈alkenyl, C₂₋₈alkynyl, cycloalkyl, aryl, heterocycle, arylC₁₋₈alkyl; heterocycloC₁₋₈alkyl; RR′N—C₁₋₈alkyl, RR′N-aryl, RO-aryl, R(O)C-aryl, RO(O)C-aryl, RR′N(O)C-aryl, —CH(α-amino acid side chain)NHR₆, —OCH₂Ph, —OCH₂-fluorenyl, —OCH₂-aryl, arylalkyloxy, —NHCH₂Ph, —NRR′ when X is other than bond;
   can form a cycle with R₂ or R₄
R₄ is chosen in the group consisting of H, α-amino acid side chain; or is C₁₋₈alkylidene when forms a cycle with R₅ and a is single bond;
R₅ is H when a is a double bond;
   or is —OR when a is a single bond;
   or can form a cycle with R₄ when R₄ is C₁₋₈alkylidene and a is single bond;
R₆ is chosen in the group consisting of —CO₂alkyl, —CO₂aryl, —SO₂aryl, —SO₂alkyl, a protecting group for amines, a peptide chain;
R is chosen in the group consisting of H, C₁₋₈alkyl, allyl, C₂₋₈alkenyl, acetyl, —C(O)—C₁₋₈alkyl; acryloyl, —C(O)—C₂₋₈alkenyl, aryl, benzyl, arylC₁₋₈alkyl, Pg;
R′ is chosen in the group consisting of C₁₋₈alkyl, benzyl, arylalkyl, allyl, C₂₋₈alkenyl, propargyl, C₂₋₈alkinyl cycloalkyl, acryloyl, —OR, —CO—C₂₋₈alkenyl, —CO—CH(α-amino acid side chain)NHR₆;
R and R′ together with N can form a cycle;
Pg is a protecting group for alcohols, amines or carboxylic acids;
the above said alkyl-, alkylidene, alkenyl-, alkynyl-, cycloalkyl-, aryl- and heterocyclic groups being able to be variably substituted.

In a preferred embodiment the present invention is related to cyclic compounds of formula (I) wherein a, X, R₃, R₅, R₆, Pg, R and R′ are as defined above and where:
i. when R₄ is α-amino acid side chain or is C₁₋₈alkylidene (optionally substituted) and forms a cycle with R₅ then
   R₁ is chosen in the group consisting of —CH₂OR, —CH₂NRR′;
   R₂ is chosen in the group consisting of —CO₂alkyl, —CH₂Oalkyl, —CH₂Oaryl, —CH₂OPg, —C(O)NRR′, —C(O)NHR′;
   R₁ and R₂ can form a cycle;
   R₂ and R₃ can form a cycle;
   R₃ and R₄ can form a cycle;
ii. when R₂ is chosen in the group consisting of —CO₂alkyl, —CH₂Oalkyl, —CH₂Oaryl, —CH₂OPg, —C(O)NRR′, —C(O)NHR′, α-amino acid side chain then R₁ is chosen in the group consisting of H, C₁₋₈alkyl, aryl, C₁₋₈alkyl, C₂₋₈alkenyl, C₂₋₈alkynyl, C₁₋₈cycloalkyl, heterocycle, arylC₁₋₈alkyl;
   heterocycloC₁₋₈alkyl;
   R₄ is H or is C₁₋₈alkylidene and forms a cycle with R₅ when a is single bond;
and wherein the above said alkyl-, alkylidene, alkenyl-, alkynyl-, cycloalkyl-, aryl- and heterocyclic groups being able to be variably substituted.

In a preferred embodiment the present invention is related to cyclic compounds of formula (VII)

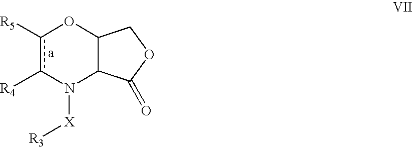

wherein:
a is a single or double bond;
X is chosen in the group consisting of “bond”, CO, SO₂;
R₃ and R₄ can form a cycle;
R₄ is α-amino acid side chain;
R₃ and R₅ are as defined above.

In a preferred embodiment the present invention is related to cyclic compounds of formula (IX)

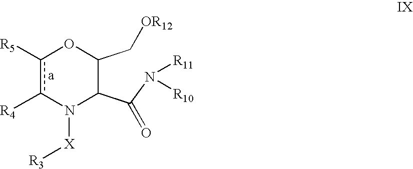

wherein:
a is a single or double bond;
X is chosen in the group consisting of CO, SO₂; bond if a is a single bond;
R₃ is as defined above;
R₃ and R₄ can form a cycle;
R₄ is α-amino acid side chain;
R₅ is as defined above, preferably chosen in the group consisting of —OH, —OCH₃, —OCH₂CH₃, —OCH(CH₃)₂ if a is a single bond; or is H if a is a double bond;
R₁₀ and R₁₁ are independently chosen in the group consisting of H, C₁₋₈alkyl, C₂₋₈alkenyl, C₂₋₈alkynyl, C₁₋₈cycloalkyl, aryl, heterocycle, arylC₁₋₈alkyl; heterocycloC₁₋₈alkyl; —OR; preferably chosen in the group consisting of H, methyl, ethyl, propyl, isopropyl, butyl, isobutyl, sec-butyl, cyclopropyl, propargyl, allyl, cyclopentyl, cyclohexyl, —OH, —OBenzyl
R₁₀ and R₁₁ can form a cycle, preferably a five, six or seven membered-ring

### R₁₂ is R

R is as defined above; preferably H, phenyl, benzyl, benzoyl, acetyl, aryl, allyl, acryloyl
said alkyl-, alkylidene, alkenyl-, alkynyl-, cycloalkyl-, aryl- and heterocyclic groups aryl being able to be variably substituted.

In a preferred embodiment the present invention is related to cyclic compounds of formula (X) or (XI)

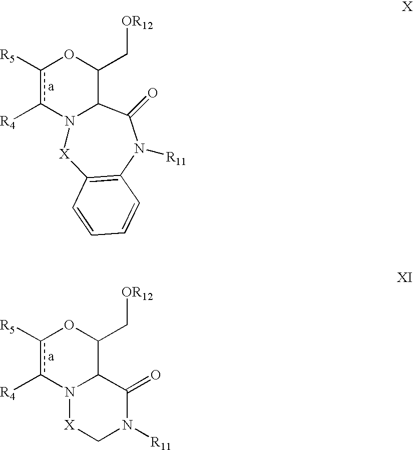

wherein:
a is a single or double bond;

### X is SO₂, CO;

R₄ is α-amino acid side chain;
R₅ is defined as above; preferably chosen in the group consisting of —OH, —OCH₃, —OCH₂CH₃, —OCH(CH₃)₂, if a is a single bond; R₅═H if a is a double bond;
R₁₁ is chosen in the group consisting of H, C₁₋₈alkyl, C₂₋₈alkenyl, C₂₋₈alkynyl, C₁₋₈cycloalkyl, aryl, heterocycle, arylC₁₋₈alkyl; heterocycloC₁₋₈alkyl; —OR; preferably chosen in the group consisting of H, methyl, ethyl, propyl, isopropyl, butyl, isobutyl, sec-butyl, cyclopropyl, propargyl, allyl, cyclopentyl, cyclohexyl, —OH, —OBenzyl;
R is as defined above;
the above said alkyl-, alkylidene, alkenyl-, alkynyl-, cycloalkyl-, aryl- and heterocyclic groups being able to be variably substituted.

In a preferred embodiment the present invention is related to cyclic compounds of formula (XII)

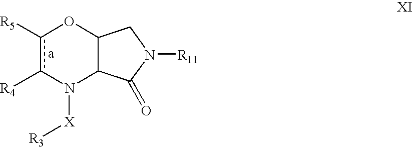

wherein:
a is a single or double bond;

### X is SO₂, CO;

R₃ is as defined above;
R₄ is α-amino acid side chain;
R₅ is defined as above; preferably —OH, —OCH₃, —OCH₂CH₃, —OCH(CH₃)₂, if a is a single bond; R₅═H if a is a double bond;
R₁₁ is chosen in the group consisting of H, C₁₋₈alkyl, C₂₋₈alkenyl, C₂₋₈alkynyl, C₁₋₈cycloalkyl, aryl, heterocycle, arylC₁₋₈alkyl; heterocycloC₁₋₈alkyl; —OR; preferably chosen in the group consisting of H, methyl, ethyl, propyl, isopropyl, butyl, isobutyl, sec-butyl, cyclopropyl, propargyl, allyl, cyclopentyl, cyclohexyl, —OH, —OBenzyl;
R is as defined above;
the above said alkyl-, alkylidene, alkenyl-, alkynyl-, cycloalkyl-, aryl- and heterocyclic groups being able to be variably substituted.

In a preferred embodiment the present invention is related to cyclic compounds of formula (VIIIa)

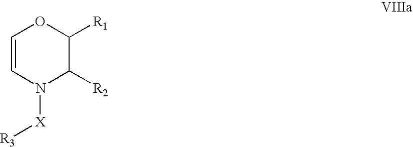

wherein
X is chosen in the group consisting of CO, SO₂;
R₁ is chosen in the group consisting of H, C₁₋₈alkyl, aryl, C₁₋₈alkyl, C₂₋₈alkenyl, C₂₋₈alkynyl, C₁₋₈cycloalkyl, heterocycle, arylC₁₋₈alkyl; heterocycloC₁₋₈alkyl; preferably chosen in the group consisting of H, methyl, ethyl, propyl, butyl, styryl, phenyl;
R₂ is chosen in the group consisting of CO₂CH₃, CO₂CH₂CH₃, CO₂CH(CH₃)₂, CH₂OCH₂Ph, CH₂OPh, CH₂OH, CH₂OPg, α-amino acid side chain;
R₃ is as defined above;
the above said alkyl-, alkylidene, alkenyl-, alkynyl-, cycloalkyl-, aryl- and heterocyclic groups being able to be variably substituted.

In a preferred embodiment the present invention is related to cyclic compounds of formula (VIIIb):

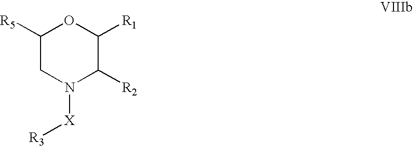

where:
X is chosen in the group consisting of “bond”, CO, SO₂;
R₁ is chosen in the group consisting of H, C₁₋₈alkyl, aryl, C₁₋₈alkyl, C₂₋₈alkenyl, C₂₋₈alkynyl, C₁₋₈cycloalkyl, heterocycle, arylC₁₋₈alkyl; heterocycloC₁₋₈alkyl preferably H, CH₃, phenyl, aryl; preferably chosen in the group consisting of H, methyl, ethyl, propyl, butyl, styryl, phenyl;
R₂ is chosen in the group consisting of CO₂CH₃, CO₂CH₂CH₃, CO₂CH(CH₃)₂, CH₂OCH₂Ph, CH₂OPh, CH₂OH, CH₂OPg, α-amino acid side chain;
R₃ is as defined above;
R₅ is defined as above; preferably chosen in the group consisting of —OH, —OCH₃, —OCH₂CH₃, —OCH(CH₃)₂;
the above said alkyl-, alkylidene, alkenyl-, alkynyl-, cycloalkyl-, aryl- and heterocyclic groups being able to be variably substituted.

In a preferred embodiment the present invention is related to cyclic compounds formula (XIII):

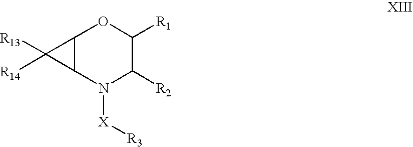

where:
X and R₃ are as defined above;
R₁ is chosen in the group consisting of H, C₁₋₈alkyl, aryl, C₁₋₈alkyl, C₂₋₈alkenyl, C₂₋₈alkynyl, C₁₋₈cycloalkyl, heterocycle, arylC₁₋₈alkyl; heterocycloC₁₋₈alkyl preferably chosen in the group consisting of H, CH₃, phenyl, aryl;
R₂ is chosen in the group consisting of CO₂CH₃, CO₂CH₂CH₃, CO₂CH(CH₃)₂, CH₂OCH₂Ph, CH₂OPh, CH₂OH, CH₂OPg, α-amino acid side chain;

### R₁₃ is H and R₁₄ is H, COOMe, COOEt, COOtBu; CH₂OH, CH₂OPg, CH₂NHPg, NHPg;

R₁₃ and R₁₄ are both halogens, preferably Cl;
the above said alkyl-, alkylidene, alkenyl-, alkynyl-, cycloalkyl-, aryl- and heterocyclic groups being able to be variably substituted.

Among pharmaceutically acceptable esters and salts within the present invention the following can be mentioned: hydrochloride, sulphate, citrate, acetate, phosphate. Also, the present invention refers to a new, useful and efficient process for the preparation of compounds as defined in formula (I) and to their use for the preparation of new compounds for therapeutic applications.

In relation to the present invention in compounds of formula (I) as above defined: protecting group (Pg) means a functional group capable of preventing the atom to which is bound to participate to an undesired reaction or to the formation of a bond, as a common strategy in chemical reactions.

Preferred functional groups are those which prevent the reaction or the binding of oxygen, nitrogen, carboxylic acids, thiols, alcohols, amines and similar groups. Such functional groups, their preparation and insertion are conventional in the state of the art, and include, for example for the reactive OH function: allyl benzyl, t-butyl, acetals, esters, trialkylsilylethers; for the COOH group: methyl, t-butyl, benzyl, phenyl, allyl, esters; for the NH group: t-Boc, Fmoc, Cbz, Alloc, Bn, Bz, Nosyl. According to the invention are included in Pg for alcohols also resins (eg. Wang resin) and sugar moieties (protected or not).

Amino acid side chain means diverse substitution as a side chain bound to an “amino acid”. The term “amino acid” includes every natural α-amino acids of the L or D series having as “side chain”: —H for glycine; —CH₃ for alanine; —CH(CH₃)₂ for valine; —CH₂CH(CH₃)₂ for leucine; —CH(CH₃)CH₂CH₃ for isoleucine; —CH₂OH for serine; —CH(OH)CH₃ for threonine; —CH₂SH for cysteine; —CH₂CH₂SCH₃ for methionine; —CH₂-(fenil) for phenylalanine; —CH₂-(phenyl)-OH for tyrosine; —CH₂-(indole) for tryptophan; —CH₂ COOH for aspartic acid; —CH₂C(O)(NH₂) for asparagine; —CH₂CH₂COOH for glutamic acid; —CH₂CH₂C(O)NH₂ for glutamine; —CH₂CH₂CH₂—N(H)C(NH₂)NH for arginine; —CH₂— (imidazole) for hystidine; —CH₂(CH₂)₃NH₂ for lysine, comprising the same side chains of amino acids bearing suitable protecting groups. According to invention the term “amino acid” includes secondary cyclic amino acids, such as proline, pipecolic, morpholine-3-carboxylic acid, thiomorpholine-3-carboxylic acid, piperazine-2-carboxylic acid and their derivatives. Moreover, the term “amino acid” includes non natural amino acids, such as ornitine (Orn), norleucine (Nle), norvaline (NVa), β-alanine, L or D α-phenylglycine (Phg), diaminopropionic acid, diaminobutyric acid, and other well known in the state of the art of peptide chemistry. In compounds of formula (I), as defined above, the groups C₁₋₈ alkyl, C₂₋₈ alkenyl and C₂₋₈ alkynyl represent linear or branched radicals, such as: methyl, ethyl, propyl, isopropyl, butyl, pentyl, hesyl, heptyl, octyl, ethenyl, propenyl, butenyl, isobutenyl, acetylenyl, propynyl, butynyl, etc. . . . .

The term cycloalkyl represents: cyclopropyl, cyclobutyl, cyclopentyl, cyclohexyl, cycloheptyl, cycloctyl, norbornyl, camphanyl, adamantyl.

The term aryl refers to the groups phenyl, biphenyl and naphtyl substituted with one or more species chosen from the groups consisting in halogens, nitrile, nitro, C₁₋₆ alkyl. The term heterocycle specifically represents: saturated or unsaturated heterocycles containing one or more nitrogen atoms, and more specifically: pyrrole, pyrazole, pyrrolidine, imidazole, indole, pyridine, pyrimidine, pyrazine, triazole, piperidine.

The term halogen represents fluoro, chloro, bromo, iodo.

The synthetic process corresponding to the present invention consists in two steps, and uses as starting compounds amino acid derivatives and glyoxal protected as acetal (step i), or amino acids and tetrose sugar derivatives (step ii), according to Scheme 1.

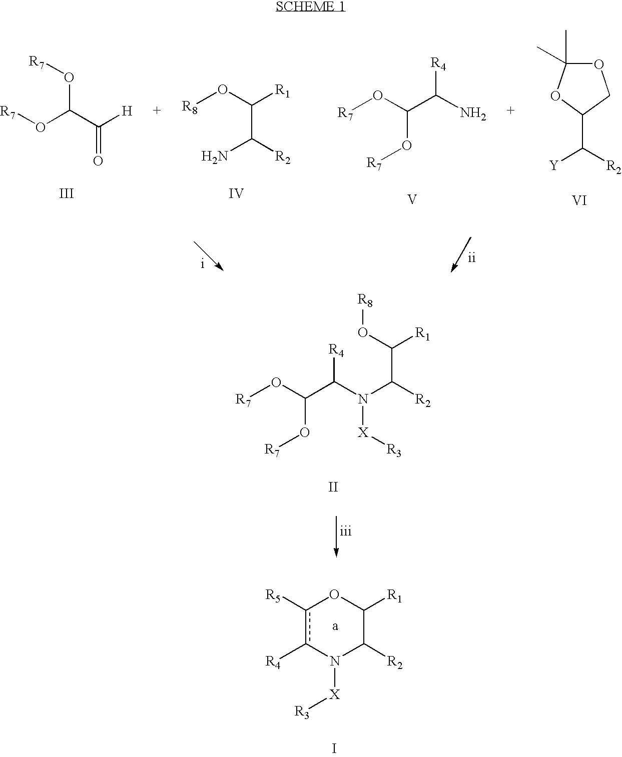

Specifically, step i consists in the condensation of glyoxal protected as acetal of formula (III)

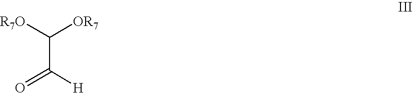

wherein R₇ is chosen in the group consisting of methyl, ethyl or the two R₇ groups form a 1,3-dioxolane or 1,3-dioxane cycle;
and a β-amino alcohol, derived from an amino acid, of formula (IV)

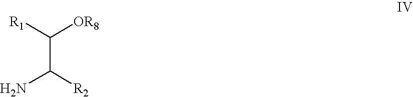

wherein
R₁ is chosen in the group consisting of H, C₁₋₈alkyl, aryl, C₁₋₈alkyl, C₂₋₈alkenyl, C₂₋₈alkynyl, C₁₋₈cycloalkyl, heterocycle, arylC₁₋₈alkyl; heterocycloC₁₋₈alkyl preferably chosen in the group consisting of preferably H, methyl, ethyl, propyl, butyl, styryl, phenyl;
R₂ is chosen in the group consisting of —CO₂alkyl, α-amino acid side chain;
R₈ is chosen in the group consisting of H, Pg;
which react in the presence of a suitable condensating agent to give compound (II)
where
X=“bond”,

### R₃═H,

R₁ is chosen in the group consisting of H, C₁₋₈alkyl, aryl, C₁₋₈alkyl, C₂₋₈alkenyl, C₂₋₈alkynyl, C₁₋₈cycloalkyl, heterocycle, arylC₁₋₈alkyl; heterocycloC₁₋₈alkyl preferably chosen in the group consisting of preferably H, methyl, ethyl, propyl, butyl, styryl, phenyl;
R₂ is chosen in the group consisting of CO₂CH₃, CO₂CH₂CH₃, CO₂CH(CH₃)₂, CH₂OCH₂Ph, CH₂OPh, CH₂OH, CH₂OPg, α-amino acid side chain,
R₇ is chosen in the group consisting of methyl, ethyl or the two R₇ groups form a 1,3-dioxolane ring;
R₈ is independently chosen in the group consisting of H, Pg; being Pg an acid-labile protecting group for alcohols preferably chosen in the group consisting of THP, TMS, TBDMS, TIS.
which can be functionalized at the nitrogen atom with suitable alkylating or acylating agents and allowed to cyclise in the presence of mineral, organic or Lewis acids to give (I).

Step ii uses a α-amino acetal, derived from an amino acid, of formula (V) or (Va)

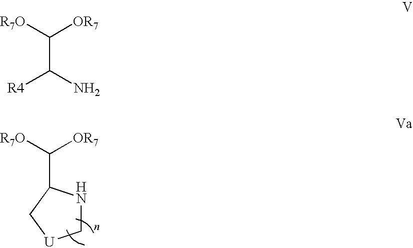

wherein
Pg is a protecting group for alcohols;
R₄ is an α-amino acid side chain;
R₇ is chosen in the group consisting of methyl, ethyl or the two R₇ groups form a 1,3-dioxolane or 1,3-dioxane cycle;

### U is —CH₂—, CH(OPg)-, CH₂—CH(OPg)-, CH(OPg)-CH₂—, —CH(NPg)-, —O—, —S—, —N(Pg)-;

n=1, 2;
Pg is a protecting group for alcohols or amines;
and a tetrose sugar derivative having the hydroxylic groups at C-3 and C-4 suitably protected and the carbon atom C-2 functionalized as a good leaving group, of formula (VI)

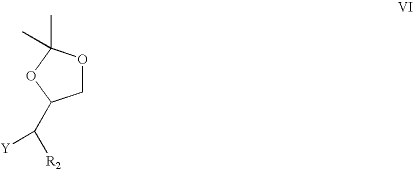

wherein
R₂ is chosen in the group consisting of —CO₂alkyl, —CH₂Oalkyl, —CH₂Oaryl, —CH₂OPg;
Y is a leaving group, preferably chosen among trifluoromethanesulfonyl, chloro, bromo, iodo, tosyl, mesyl, trichloromethanesulfonyl; to give compound (II) where
X=“bond”,

### R₂ is CO₂CH₃, CO₂CH₂CH₃;

### R₃═H,

R₄ is a α-amino acid side chain;
R₃ and R₄ can form a cycle if a secondary amino acid derivative Va is used
R₇ is chosen in the group consisting of methyl, ethyl or the two R₇ groups form a 1,3-dioxolane or 1,3-dioxane cycle;
R₁ and R₈ can form a cycle, preferably 1,3-dioxolane cycle optionally substituted;
Specifically, the process corresponding to the present invention allows the preparation of compounds of formula (I), where:

If step i is chosen:

R₁ is chosen in the group consisting of H, C₁₋₈alkyl, aryl, C₁₋₈alkyl, C₂₋₈alkenyl, C₂₋₈alkynyl, C₁₋₈cycloalkyl, heterocycle, arylC₁₋₈alkyl; heterocycloC₁₋₈alkyl;
R₂ is chosen in the group consisting of —CO₂alkyl, —CH₂Oalkyl, —CH₂Oaryl, —CH₂OPg, —C(O)NRR′, —C(O)NHR′, α-amino acid side;
R₄ is H or is C₁₋₈alkylidene (optionally substituted) and forms a cycle with R₅ when a is single bond;
a, X, R₃, R₅, R₆, R and R′ are as defined above.

If step ii is chosen:

R₁ is chosen in the group consisting of —CH₂OR, —CH₂NRR′,
R₂ is chosen in the group consisting of —CO₂alkyl, —CH₂Oalkyl, —CH₂Oaryl, —CH₂OPg, —C(O)NRR′, —C(O)NHR′;
R₁ and R₂ can form a cycle, preferably a γ-lactone;
R₂ and R₃ can form a cycle;
R₃ and R₄ can form a cycle;
R₄ is α-amino acid side chain; or is C₁₋₈alkylidene and forms a cycle with R₅
a, X, R₃, R₅, R₆, R and R′ are as defined above;

Suitably protected glyoxal derivatives (III) are commercially available or easily preparable according to procedures known in the state of the art. β-Amino alcohol (IV), where R₁ is H and R₂ is an amino acid side chain, is commercially available or can be easily prepared starting from α-amino acids by reduction of the corresponding methylester derivative, according to procedures known in the state of the art. If R₁=alkyl, or aryl, compound (IV) can be achieved according to procedures known in the state of the art, for example by alkylation of the ester using an organometallic reagent, followed by reduction of the resulting ketone to the corresponding alcohol, or from hydrolysis of the suitable oxazolines resulting from reaction of methyl isocyanoacetate with aldehydes in the presence of a chiral catalyst, according to procedures known in the state of the art (for the preparation of oxazolines, see: Ito, Y. et al., J. Am. Chem. Soc. 1986, 108, 6405; Panella, L. et al., J. Org. Chem. 2006, 71, 2026; Hughes, P. F. et al., Eur. J. Org. Chem. 1994, 5799).

The condensation of (III) with (IV), where R₈ can be H or Pg, where Pg is preferably acid-labile, and preferably chosen in the group consisting of TMS, TBDMS, TIS, THP, to give compound (II), where X=“bond” and R₃ is H, is preferably obtained by reductive amination in a protic solvent, in particular methanol or ethanol, using a reducing agent, preferably hydrogen over palladium as catalyst. If R₁ is not H, R₈ cannot be H, but necessarily is a protecting group for alcohols (Pg). Moreover, such group has to be necessarily removed before the functionalization of the nitrogen atom so as to reduce the local steric hindrance which does not allow such reaction on the nitrogen atom. The process of protecting group (Pg) removal can lead to cyclization of the intermediate (II) to give compound (I), where X=“bond” and R₃═H, according to the process iii, as in Scheme 1 described next, and such compound can be successively functionalized at the nitrogen atom to give product (I) having a=double bond, X different form “bond” and R₃ different from H, thus allowing in the process iii of Scheme 1 the inversion of the processes of nitrogen atom functionalization and cyclization to give the morpholine nucleus of structure (I).

The intermediate (II) is functionalized at the nitrogen atom through processes of alkylation and acylation known in the state of the art, so as to insert preferably an alkyl, aryl, amide, urethane, sulfonamide, urea, thioamide, and thiourea group. Compound (II) is successively cyclized to give the final product (I) using an acid, which allow the ketalization of the hydroxylic function deriving from compound (IV) on the aldehyde moiety belonging to (III) and protected as an acetal. The synthetic step iii and the reaction conditions are important, because if a polar protic solvent, preferably chosen between water, methanol, ethanol and isopropanol, in the presence of acid catalysis obtained by adding HCl or SOCl₂, the emiacetal of structure (I), where a=single bond and R₅═OH, or the acetal of structure (I), where a=single bond and R₅═OR, where R is H, an alkyl (depending from the used solvent) is obtained; in an apolar aprotic solvent, preferably benzene or toluene, in the presence of acid catalysis, obtained preferably with p-toluenesulfonic acid or sulfuric acid adsorbed on silica gel, and in the presence of molecular sieves, heating at the refluxing temperature until reaction completion (typically 2 h), compound (I) is achieved where a=double bond and R₅═H. Moreover, if compound (II) is functionalized at the nitrogen atom with an alkyl or aryl group, specifically X=“bond” and R₃ is an alkyl or aryl, according to processes known in the state of the art, such as nucleophilic substitution, reductive amination, or arylation catalyzed preferably by Pd or Cu complexes, compound (I) is obtained where a=single bond and R₅═OR, being R an alkyl group.

In the process of reaction ii, starting compound (V) is commercially available or can be obtained in few steps through known procedures for some acetals and easily applicable to the others (Barany, G. e altri Tetrahedron Lett. 2000, 41, 6131-6135; Williams, R. S. e altri J. Am. Chem. Soc. 2003, 125, 8561-8565); in particular, starting from amino acids protected at the nitrogen atom, preferably with Fmoc, Cbz, or Boc, COOH group is transformed in aldehyde in two steps according to procedures known in the state of the art, and successively protected as acetal, linear or cyclic, using a polar protic solvent, preferably methanol, ethanol or ethanediol, and in the presence of an acid in catalytic quantities, preferably p-toluenesulfonic acid, an acidic ion-exchange resin, or sulfuric acid adsorbed on silica gel. Finally, the amino group is deprotected to give compound (V). The intermediate compound (VI) can be achieved by functionalization at C-2, as previously reported (Trabocchi, A. et al. Synthesis 2006, 3122-3126), of a commercially available derivative, or easily preparable from ascorbic acid in few steps and high yields according to procedures reported in the literature (Sasaki, A. N. et al. J. Org. Chem. 2006, 71, 693-703). Successively, reaction (V) with (VI) through nucleophilic substitution in a polar aprotic solvent, preferably dichloromethane or chloroform, in the presence of a base, preferably ethyldiisopropylamine, triethylamine, pyridine or 2,6-lutidine, at 20-30° C. for at least 18 h and in any case until complete conversion of starting material. The process of functionalization of the amino group and of cyclization by acetalization of compound (II) (process iii) can be changed in the order, in agreement with the chemical functions present in R₂ and R₄. The process of acetalization to give (I), where a=single bond and R₅═OR, where R═H, alkyl (depending from the solvent used), preferably H, methyl, ethyl, isopropyl, is carried out on compound (II) as above described in a polar protic solvent, preferably chosen between water, methanol, ethanol, isopropanol, and in the presence of an acid catalyst, preferably chosen between p-toluenesulfonic acid, an acidic ion-exchange resin, or sulfuric acid adsorbed on silica gel. It is strictly required to carry out the acetalization process on compound (II) before the functionalization of its amino group if:

R₄ is a side chain containing a functional group belonging to the class of amines, alcohols and carboxylic acids, protected with an acid-labile group, X═CO, and R₃═—Oalkyl, —Oaryl;
R₃ contains an olefinic group.

Moreover, if step iii is carried out on compound (II), where X is not a bond, in an apolar aprotic solvent, preferably benzene or toluene, in the presence of an acid catalyst, preferably p-toluenesulfonic acid, sulfuric acid adsorbed on silica gel, and in the presence of molecular sieves, heating at the refluxing temperature until reaction completion (typically 2 h), compound (I) is achieved where a=double bond and R₅═H. Moreover, by using this reaction conditions compounds of general formula (I), where a is a double bond and R₅ is H, are obtained starting from the corresponding molecules VII, where X is not a bond.

When compound (Va) is used, which derives from a secondary cyclic amino acid, such as proline, pipecolic, morpholine-3-carboxylic acid, thiomorpholine-3-carboxylic acid, piperazine-2-carboxylic acid and their derivatives, prepared according to the state of the art (see for example: Mori, S. et al., Tetrahedron 1991, 47, 5051; Trabocchi, A. et al., Tetrahedron Lett. 2005, 46, 7813; Watkins, W. J., et al., Bioorg. Med. Chem. 2003, 13, 4241), then R₄ and R₃ of compound (II) and (I) can form a cycle according to steps ii and iii, as shown in Scheme 2.

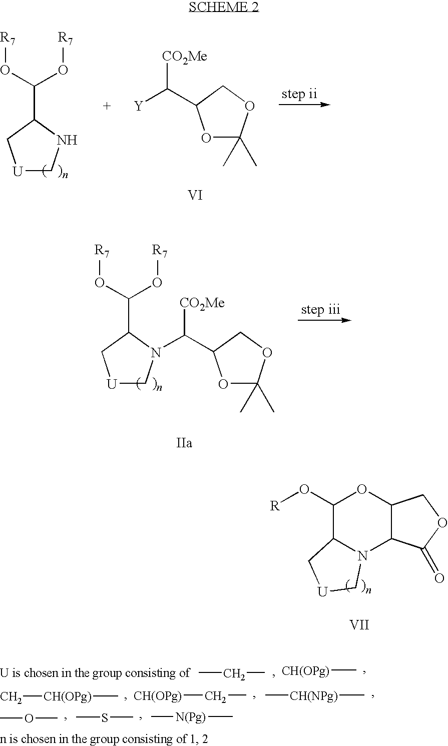

The synthetic process of the present invention, through step ii, allows to obtain specifically molecules of formula (VII):

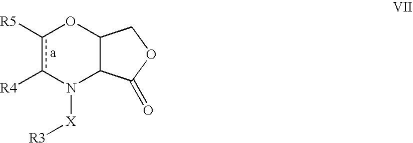

wherein:
a is a single or double bond;
X is chosen in the group consisting of “bond”, CO, SO₂;
R₃ is as defined above
R₄ is an α-amino acid side chain
R₃ and R₄ can form a cycle;
R₅ is H when a is a double bond;
is chosen in the group consisting of OR when a is a single bond; form a cycle with R₄ when R₄ is methylene optionally substituted;
R and R′ are as defined above;

The synthetic process through step i allows to achieve in particular compounds of formula (VIII):

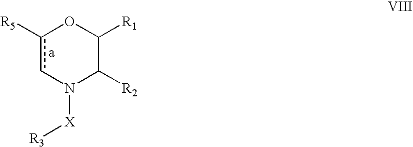

where:
a is a single or double bond;
X is preferably chosen in the group consisting of CO, SO₂, a bond only if a is a single bond;
R₁ is preferably chosen in the group consisting of H, CH₃, aryl;
R₂=—CO₂alkyl, —CH₂Oalkyl, —CH₂Oaryl, —CH₂OH, —CH₂OPg, α-amino acid side chain;
R₅ is —OR if a is a single bond, or R₅ is H if a is a double bond;
R₃ and R are defined as above;
the aryl being able to be variably substituted, preferably with F, Cl, Br, I.

Moreover, molecules of formula (VII), prepared according to the process of the present invention, allow the achievement of further compounds of formulae (IX)-(XII), still containing the morpholine nucleus of structure (I) through processes known in the state of the art, and as reported in Scheme 7.

Molecules of formula (VIIIa), prepared according to the process of the present invention, allow to achieve further molecules of structure (XIII), still containing the morpholine nucleus of structure (I) through processes known in the literature, and reported in the Scheme 8.

Also, compounds of general formula I where a=single bond and R₅═—OH, and R₁, R₂, X, R₃, R₄ are defined as above and preferably compounds of formula VII where R₅═—OH and X, R₃, R₄ are defined as above, can be used for obtaining other morpholine-based heterocycles of general formula I where R₅═—Oalkyl, —Ocycloalkyl, —Oalkenyl, —Oalkynyl, —O-resin, for example according to the following procedure:

Compound I (where a=single bond, R₅═H, and R₁, R₂, X, R₃, R₄ are defined as above) can be treated with trichloroacetonitrile in the presence of a suitable base, preferably DBU (1,8-Diazabicyclo[5.4.0]undec-7-ene) to give the corresponding trichloroacetimidate, which in turn can react with alcohols or Wang and HMBA resins in anhydrous solvents, preferably dichloromethane-cyclohexane mixture, in the presence of catalytic quantities of a Lewis acid, preferably BF₃.Et₂O, to give the corresponding molecules of general formula VII where R₅ is as defined above (see Examples 28-31).

Compounds of general formula VII, where a=single bond, R₄═—CH₂—COOMe or —CH₂—CH₂—COOMe, X═CO, R₃═—CH(α-amino acid side chain)NH-Pg and Pg and R are as defined above, can be treated according to procedures known in the literature, for example when Pg=Boc consisting in treatment with TFA followed by work-up and heat in DMF in the presence of a base, to give compounds of formulae VIIIa and VIIb, as shown in Scheme 3:

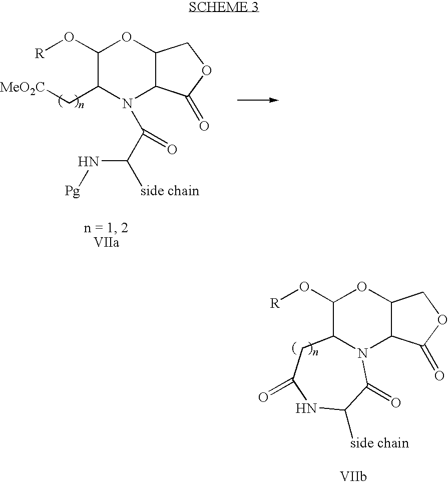

### EXPERIMENTAL DETAILS

The present invention is better understood in view of the following examples. In particular, examples 1-18 demonstrate that according to Scheme 4, if V and VI are reacted according to step ii, compound II is obtained, which in turn gives compound VII through step iii:

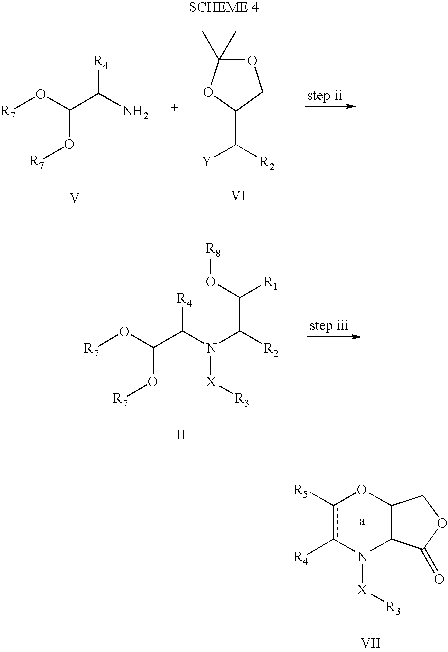

### Example 1

### Synthesis of (5R/S,3aS,7aR)-7-Benzoyl-5-methoxy-hexahydro-2,4-dioxa-7-aza-inden-1-one [structure VII where a=single bond, X═CO, R₃═Ph, R₄═H, R₅═OCH₃]

A solution of V (where R₄═H, R₇═CH₃) (150 mg, 0.79 mmol) in dry CH₂Cl₂ (1.4 mL) was cooled to −10° C., and precooled dry pyridine (135 μL) was added, then a solution of trifluoromethanesulfonic anhydride (219 μL, 1.02 mmol), corresponding to VI (where R₂═COOMe, Y═OTf) in dry CH₂Cl₂ (0.40 mL) was added over 30 min. The mixture was stirred at room temperature for 30 min and then neutralized with a saturated NaHCO₃ solution. The organic layer was separated, dried over Na₂SO₄, filtered and concentrated in vacuo to give a dark oil. Flash chromatography (Petroleum ether—EtOAc, 2:1, R_{f}=0.6) afforded II (a=single bond, X=bond, R₃═H, R₁ and R₈═—C(CH₃)₂—OCH₂—, R₂═COOMe, R₄═H, R₇═CH₃) as a yellow oil (136 mg, 53%). ¹H NMR (200 MHz, CDCl₃): δ=5.24 (d, J=3.6 Hz, 1H, CF₃SO₂OCH), 4.58-4.50 (m, 1H, >CHO), 4.10-3.94 (m, 2H, CH₂O), 3.84 (s, 3H, OCH₃), 1.43 (s, 3H, CH₃), 1.34 (s, 3H, CH₃). ¹³C NMR (50 MHz, CDCl₃): δ=165.0 (s, C═O), 121.5 (s, CF₃), 110.9 (s, C(CH₃)₂), 81.0 (d, CF₃SO₂OCH), 74.0 (d, >CHO), 65.4 (t, CH₂), 53.5 (q, O CH₃), 25.8 (q, OCH₃), 24.9 (q, CH₃). MS m/z (%) 322 (M⁺, 3), 75 (100), 55 (62). To a solution of compound II (where X=bond, R₃═H, R₁ and R₈═—C(CH₃)₂—OCH₂—, R₂═COOMe, R₄═H, R₇═CH₃) (1 eq) and DIPEA (1.2 eq) in anhydrous THF (1.8 mL/mmol) benzoyl chloride (1 eq) is added at 0° C. The mixture is allowed to reach room temperature and is left overnight stirring under N₂. Successively the mixture is concentrated, diluted with EtOAc, and washed with water and brine. The organic phase is dried over Na₂SO₄ and concentrated. Compound II (where a=single bond, X═CO, R₁ and R₈═—C(CH₃)₂—OCH₂—, R₂═COOMe, R₃═Ph, R₄═H, R₇═CH₃) is isolated by flash chromatography (petroleum ether—EtOAc, 1:1). Colourless oil, yield: 99%. ¹H NMR (200 MHz, CDCl₃): mixture of rotamers δ=7.50-7.41 (m, 5H), 4.79 (br, 1H), 4.47 (br, 2H), 4.12-3.96 (m, 2H), 3.74 (s, 3H), 3.46-3.43 (m, 2H), 3.33 (s, 3H), 3.23 (s, 3H), 1.32 (s, 6H).

A solution of II (where X═CO, R₁ and R₈═—C(CH₃)₂—OCH₂—, R₂═COOMe, R₃═Ph, R₄═H, R₇═CH₃) and SOCl₂ (1.5 eq) in MeOH (10 mL/mmol) is overnight stirred at room temperature under N₂. Successively, the mixture is concentrated and purified by flash chromatography (petroleum ether—EtOAc, 1:1), thus giving product VII (where a=single bond, X═CO, R₃═Ph, R₄═H, R₅═OCH₃) as a white solid, yield: 66%. ¹H NMR (400 MHz, CDCl₃): mixture of epimers and rotamers δ=7.60-7.53 (m, 2H), 7.47-7.42 (m, 3H), 5.68 (d, 1H), 4.66-4.28 (m, 4H), 3.67 (d, 1H major), 3.46 (s, 3H minor), 3.41 (s, 3H major), 3.30 (d, 1H major), 2.99 (d, 1H minor). ¹³C NMR (50 MHz, CDCl₃): mixture of epimers and rotamers δ=171.9-171.7 (s, 2C), 133.5-133.3 (s, 1C), 130.3-130.1 (d, 1C), 128.4-128.2 (d, 2C), 127.2-126.9 (d, 2C), 95.3-94.7 (d, 1C), 70.8-70.5 (t, 1C), 65.3-64.9 (d, 1C), 56.5-56.1 (q, 1C), 51.3 (d, 1C), 47.2 (t, 1C).

### Example 2

### Synthesis of (5R/S,3aS,7aR)-7-carbobenzyloxy-5-methoxy-hexahydro-2,4-dioxa-7-aza-inden-1-one [structure VII where a=single bond, X═CO, R₃═OCH₂Ph, R₄═H, R₅═OCH₃]

To a solution of II (where X=bond, R₃═H, R₁ and R₈═—C(CH₃)₂—OCH₂—, R₂═COOMe, R₄═H, R₇═CH₃) (1 eq) and NaHCO₃ (2 eq) in H₂O-EtOAc (1.7 mL/mmol-2 mL/mmol) Cbz-Cl (1 eq) is added at 0° C. The mixture is allowed to reach room temperature and is left overnight stirring under N₂. Successively, the mixture is washed with aqueous 1N HCl and brine. The organic phase is dried over Na₂SO₄ and concentrated. Compound II (where X═CO, R₁, and R₈═—C(CH₃)₂—OCH₂—, R₂═COOMe, R₃═OCH₂Ph, R₄═H, R₇═CH₃) is obtained after flash chromatography (petroleum ether—EtOAc, 1:1) as a colourless oil, yield: 80%. ¹H NMR (200 MHz, CDCl₃): mixture of rotamers δ=7.35-7.21 (m, 5H), 5.18-5.14 (m, 1H), 4.70-4.42 (m, 3H), 4.04-3.85 (m, 2H), 3.73-3.56 (m, 4H), 3.60 (s, 3H), 3.41-3.27 (m, 6H), 1.39 (s, 3H), 1.35 (s, 3H).

A solution of II (where X═CO, R₁ ed R₈═—C(CH₃)₂—OCH₂—, R₂═COOMe, R₃═OCH₂Ph, R₄═H, R₇═CH₃) and SOCl₂ (1.5 eq) in MeOH (10 mL/mmol) is overnight stirred at room temperature under N₂. Successively, the mixture is concentrated and purified by flash chromatography (petroleum ether—EtOAc, 1:1), giving product VII (where a=single bond, X═CO, R₃═OCH₂Ph, R₄═H, R₅═OCH₃) as a white solid, yield: 99%. ¹H NMR (200 MHz, CDCl₃): mixture of epimers and rotamers δ=7.36 (s, 5H), 5.21 (s, 2H), 4.85-4.60 (m, 2H), 4.48-4.36 (m, 3H), 4.08-3.94 (m, 1H), 3.44 and 3.41 (s, 3H), 3.14-2.99 (m, 1H). ¹³C NMR (50 MHz, CDCl₃): mixture of epimers and rotamers δ=172.3 (s, 1C), 155.9-155.3 (s, 1C), 135.9 (s, 1C), 128.5 (d, 2C), 128.1 (d, 2C), 127.7 (d, 1C), 95.7-95.2 (d, 1C), 71.2 (t, 1C), 68.2 (t, 1C), 65.3-65.0 (d, 1C), 55.7 (q, 1C), 53.9-53.4 (d, 1C), 44.1-43.4 (t, 1C).

### Example 3

### Synthesis of (5R/S,3aS,7aR)-5-methoxy-7-(2-nitrobenzyl)-hexahydro-2,4-dioxa-7-aza-inden-1-one [structure VII where a=single bond, X═CO, R₃═OCH₂(2-NO₂)Ph, R₄═H, R₅═OCH₃]

To a solution of II (where X=bond, R₃═H, R₁ and R₈═—C(CH₃)₂—OCH₂—, R₂═COOMe, R₄═H, R₇═CH₃) (1 eq) and 2-nitrobenzaldehyde (1 eq) in THF (0.2M) NaBH(OAc)₃ (1.3 eq) is added in small portions. The mixture is left overnight stirring at room temperature, then it is concentrated, diluted with EtOAc, and washed with water and brine. The organic phase is dried over Na₂SO₄ and concentrated. Compound II (where X═CO, R₁ ed R₈═—C(CH₃)₂—OCH₂—, R₂═COOMe, R₃═-CH₂(2-NO₂)Ph, R₄═H, R₇═CH₃) is obtained after flash chromatography (petroleum ether—EtOAc, 1:1) as a colourless oil, yield: 40%. ¹H NMR (200 MHz, CDCl₃): mixture of epimers δ=8.14 (d, 1H), 7.78-7.62 (m, 2H), 7.49-7.38 (m, 2H), 4.96 (s, 2H), 4.38-4.29 (m, 1H), 4.11-3.98 (m, 1H), 3.77 (s, 3H), 3.24 (s, 3H), 3.19 (s, 3H), 2.91-2.57 (m, 2H), 1.24 (s, 6H).

A solution of II (where X═CO, R₁ and R₈═—C(CH₃)₂—OCH₂—, R₂═COOMe, R₃═-CH₂(2-NO₂)Ph, R₄═H, R₇═CH₃) and SOCl₂ (1.5 eq) in MeOH (10 mL/mmol) overnight stirred at room temperature and under N₂. Successively, the mixture is concentrated and purified through flash chromatography (petroleum ether—EtOAc, 1:1), thus giving compound VII (where a=single bond, X═CO, R₃═—CH₂(2-NO₂)Ph, R₄═H, R₅═OCH₃) as an oil, yield: 43%. ¹H NMR (200 MHz, CDCl₃): mixture of epimers δ=7.88-7.38 (m, 4H), 4.62 (m, 1H), 4.60 (d, J=15.4 Hz, 1H), 4.5 (m, 1H), 4.32 (d, J=15.4 Hz, 1H), 4.35 (d, J=8 Hz, 1H), 4.25 (s, 2H), 3.59 (d, J=4.0 Hz, 1H), 3.38 (s, 3H), 2.83 (dd, J=12.5, 2.5 Hz, 1H), 2.60 (d, J=12.5 Hz, 1H).

### Example 4

### Synthesis of (5R/S,3aS,7aR)-5-methoxy-7-acetyl-hexahydro-2,4-dioxa-7-aza-inden-1-one [structure VII where a=single bond, X=bond, R₃═CH₃, R₄═H, R₅═OCH₃]

To a solution of II (where X=bond, R₃═H, R₁ and R₈═—C(CH₃)₂—OCH₂—, R₂═COOMe, R₄═H, R₇═CH₃) (1 eq) and DIPEA (3.5 eq) in anhydrous CH₂Cl₂ (2 mL/mmol) Ac₂O (3 eq) and DMAP (0.1 eq) are added. The mixture is left overnight stirring under N₂. Successively the mixture is washed with H₂O/ice and 1M KHSO₄. The organic phase is dried over Na₂SO₄ and concentrated. Compound II (where a=single bond, X=bond, R₃═CH₃, R₁ and R₈═—C(CH₃)₂—OCH₂—, R₂═COOMe, R₄═H, R₇═CH₃) is isolated by flash chromatography (petroleum ether—EtOAc, 1:1) as a colourless oil; yield: 95%. ¹H NMR (200 MHz, CDCl₃): mixture of epimers and rotamers δ=4.78-4.71 (m, 1H), 4.52 (t, 1H), 4.34 (d, 1H), 4.08-4.01 (d, 1H), 3.88-3.80 (m, 1H), 3.73 (s, 3H), 3.55 (d, 1H), 3.46 (s, 3H), 3.44 (s, 3H), 3.36 (d, 1H), 2.16 (s, 3H), 1.39 (s, 3H), 1.34 (s, 3H).

A solution of II (where X=bond, R₃═CH₃, R₁ and R₈═—C(CH₃)₂—OCH₂—, R₂═COOMe, R₄═H, R₇═CH₃) and SOCl₂ (1.5 eq) in MeOH (10 mL/mmol) is overnight stirred at room temperature under N₂. Successively the mixture is concentrated and purified by flash chromatography (petroleum ether—EtOAc, 1:1) thus giving product VII (where a=single bond, X═CO, R₃═CH₃, R₄═H, R₅═OCH₃) as a yellow oil; yield: 72%. ¹H NMR (200 MHz, CDCl₃): mixture of epimers and rotamers δ=5.55 (d, 1H), 4.65 (s, 1H), 4.43-4.24 (m, 3H), 3.64 (d, 1H), 3.36 (s, 3H), 3.28 (dd, 1H), 2.16 (s, 3H). ¹³C NMR (50 MHz, CDCl₃): mixture of epimers and rotamers δ=172.3 (s, 1C), 171.7 (s, 1C), 95.3-94.6 (d, 1C), 70.7 (t, 1C), 65.3-64.3 (d, 1C), 55.2-55.0 (q, 1C), 51.0 (d, 1C), 46.1 (t, 1C), 20.3 (q, 1C).

### Example 5

### Synthesis of (5R/S,3aS,7aR)-5-methoxy-hexahydro-2,4-dioxa-7-aza-inden-1-one [structure VII where a=single bond, X=bond, R₃═H, R₄═H, R₅═OCH₃]

Compound II (where X=bond, R₃═H, R₁ and R₈═—C(CH₃)₂—OCH₂—, R₂═COOMe, R₄═H, R₇═CH₃) (1 eq) is added to a solution of SOCl₂ (2.5 eq) in MeOH (5 mL/mmol). The mixture is refluxed for 4 h under N₂. Successively the mixture is concentrated and filtered on a weakly basic resin giving quantitatively compound VII (where a=single bond, X=bond, R₃═H, R₄═H, R₅═OCH₃) as a yellow oil. ¹H NMR (200 MHz, CDCl₃): mixture of epimers δ=4.43-4.28 (m, 3H), 3.76-3.70 (m, 2H), 3.45 (s, 3H major),3.40 (s, 3H minor), 2.94-2.76 (m, 2H), 2.44 (br, 1H). ¹³C NMR (50 MHz, CDCl₃): mixture of epimers δ=174.8 (s, 1C), 95.8 (d, 1C), 70.8 (t, 1C), 65.3 (d, 1C), 55.6 (q, 1C), 54.8 (d, 1C), 44.4 (t, 1C).

### Example 6

### Synthesis of (5R/S,3aS,7aR)-6-carbomethoxymethyl-5-methoxy-hexahydro-2,4-dioxa-7-aza-inden-1-one [structure VII where a=single bond, X=bond, R₃═H, R₄═CH₂COOMe, R₅═OCH₃]

A solution of VI (where R₂═COOMe, Y═OTf) (130 mg, 0.4 mmol) in dry CH₂Cl₂ (5 mL) was cooled to 0° C. under N₂, then a solution of V (where W═H, R₄═CH₂COOtBu, R₇═CH₃) (110 mg, 0.5 mmol) and DIPEA (0.14 mL, 0.8 mmol) in dry CH₂Cl₂ (3 mL) were added. The mixture was stirred at room temperature for 15 h, then it was extracted with a saturated NaHCO₃ solution. The organic layer was dried over Na₂SO₄, filtered and concentrated in vacuo to give a dark oil. Flash chromatography afforded pure II (where X=bond, R₁ and R₂═—C(CH₃)₂—OCH₂—, R₃═H, R₄═CH₂COOtBu, R₇═CH₃) as a yellow oil (133 mg, 85%). ¹H NMR (200 MHz, CDCl₃): δ=4.17 (d, 1H), 4.12-4.07 (m, 1H), 3.99 (d, 2H), 3.73 (s, 3H), 3.48 (d, 1H), 3.38 (s, 3H), 3.36 (s, 3H), 3.18-3.09 (m, 1H), 2.45 (dd, 1H), 2.23 (dd, 1H), 2.03 (br, 1H), 1.43 (s, 9H), 1.40 (s, 3H), 1.30 (s, 3H). ¹³C NMR (50 MHz, CDCl₃): δ=173.4 (s, 1C), 170.8 (s, 1C), 109.4 (s, 1C), 106.8 (d, 1C), 80.4 (s, 1C), 77.1 (d, 1C), 66.9 (t, 1C), 62.1 (d, 1C), 55.2 (q, 3C), 51.8 (d, 1C), 33.6 (t, 1C), 28.1 (q, 3C), 26.7 (q, 1C), 25.2 (q, 1C).

Compound II (where X=bond, R₃═H, R₁ and R₈═—C(CH₃)₂—OCH₂—, R₂═COOMe, R₄═CH₂COOtBu, R₇═CH₃) (1 eq) is added to a solution of SOCl₂ (2.5 eq) in MeOH (5 mL/mmol). The mixture is refluxed for 4 h under N₂. Successively the mixture is concentrated and filtered on a weakly basic resin giving compound VII (where a=single bond, X=bond, R₃═H, R₄═CH₂COOMe, R₅═OCH₃) as a yellow oil. ¹H NMR (200 MHz, CDCl₃): mixture of epimers δ=4.49-4.13 (m, 3H), 3.64 (s, 3H minor), 3.59 (m, 3H major), 3.53-3.41 (m, 3H), 3.35 (s, 3H major), 3.33 (s, 3H minor), 3.16-3.02 (m, 1H), 2.57-2.32 (m 2H). ¹³C NMR (50 MHz, CDCl₃): mixture of epimers δ=176.5-173.1 (s, 1C), 172.1-171.6 (s, 1C), 106.6 (d, 1C), 98.9 (d, 1C), 71.8 (d, 1C), 70.8 (t, 1C), 64.7-62.9 (d, 1C), 63.8-62.7 (t, 1C), 55.4-55.9 (q, 1C), 52.5-52.2 (q, 1C).

### Example 7

### Synthesis of (5R/S,3aS,7aR)-7-(2-Bromo-acetyl)-5-methoxy-hexahydro-2,4-dioxa-7-aza-inden-1-one [structure VII where a=single bond, X═CO, R₃═CH₂Br, R₄═H, R₅═OCH₃]

To a solution of VII (where a=single bond, X=bond, R₃═H, R₄═H, R₅═OCH₃) (1 eq) and TEA (1 eq) in anhydrous CH₂Cl₂ (1 mL/mmol) bromoacetyl bromide (1 eq) is added dropwise at 0° C. The mixture is allowed to reach room temperature and is left 30 min stirring under N₂. Successively the mixture is diluted with H₂O, washed with 1N HCl and brine. The organic phase is dried over Na₂SO₄ and concentrated. Compound VII (where a=single bond, X═CO, R₃═CH₂Br, R₄═H, R₅═OCH₃) is isolated by flash chromatography (petroleum ether—EtOAc, 1:1). White solid, yield: 55%. ¹H NMR (200 MHz, CDCl₃): mixture of epimers and rotamers δ=5.44 (d, 1H major), 5.34 (d, 1H, minor), 4.74-4.22 (m, 5H), 3.93 (d, 1H major), 3.90 (d, 1H minor), 3.67 (d, 1H major), 3.48 (d, 1H minor), 3.39 (s, 3H), 3.33 (dd, 1H major), 2.81 (dd, 1H minor). ¹³C NMR (50 MHz, CDCl₃): mixture of epimers and rotamers δ=172.1 (s, 1C), 167.1 (s, 1C), 95.8-94.8 (d, 1C), 70.8-70.7 (t, 1C), 65.7-64.8 (d, 1C), 55.5 (q, 1 C), 51.6 (d, 1C), 46.6 (t, 1C), 25.7-25.4 (t, 1C).

### Example 8

### Synthesis of (5R/S,3aS,7aR)-7-(2-nitrobenzoyl)-5-methoxy-hexahydro-2,4-dioxa-7-aza-inden-1-one [structure VII where a=single bond, X═CO, R₃=2-nitrophenyl, R₄═H, R₅═OCH₃]

To a solution of VII (where a=single bond, X=bond, R₃═H, R₄═H, R₅═OCH₃) (1 eq) and TEA (1.5 eq) in anhydrous CH₂Cl₂ (2.5 mL/mmol) a solution of 2-nitrobenzoyl chloride (1.2 eq) in anhydrous CH₂Cl₂ (2.5 mL/mmol) is added at 0° C. The mixture is allowed to reach room temperature and is left overnight stirring under N₂. Successively the mixture is washed with NaHCO₃, 1N HCl and brine. The organic phase is dried over Na₂SO₄ and concentrated. Compound VII (where a=single bond, X═CO, R₃=2-nitrophenyl, R₄═H, R₅═OCH₃) is isolated by flash chromatography (petroleum ether—EtOAc, 1:2). White solid, yield: 83%. ¹H NMR (200 MHz, CDCl₃): mixture of epimers and rotamers δ=8.15 (d, 1H major), 8.07 (d, 1H minor), 7.74-7.47 (m, 3H), 5.59 (d, 1H), 4.79 (s, 1H minor), 4.63-4.23 (m, 4H), 4.35 (d, 1H), 3.39 (s, 3H minor), 3.35 (s, 3H major), 3.24-3.18 (m, 1H major), 2.96 (dd, 1H minor). ¹³C NMR (50 MHz, CDCl₃): mixture of epimers and rotamers δ=173.9 (s, 1C), 167.7 (s, 1C), 134.8 (s, 1C), 134.5-134.2 (d, 1C), 130.4 (d, 1C), 128.7 (d, 1C), 124.7 (d, 1C), 116.1 (d, 1C), 95.6-94.8 (d, 1C), 70.9 (t, 1C), 65.5-65.0 (d, 1C), 56.5-55.6 (q, 1C), 51.4 (d, 1C), 46.8 (t, 1C).

### Example 9

### Synthesis of (5R/S,3aS,7aR)-7-(2-iodobenzoyl)-5-methoxy-hexahydro-2,4-dioxa-7-aza-inden-1-one [structure VII where a=single bond, X═CO, R₃=2-iodophenyl, R₄═H, R₅═OCH₃]

To a solution of VII (where a=single bond, X=bond, R₃═H, R₄═H, R₅═OCH₃) (1 eq) and TEA (1.5 eq) in anhydrous CH₂Cl₂ (2.5 mL/mmol) a solution of 2-iodobenzoyl chloride (1.2 eq) in anhydrous CH₂Cl₂ (2.5 mL/mmol) is added at 0° C. The mixture is allowed to reach room temperature and is left overnight stirring under N₂. Successively the mixture is washed with NaHCO₃, 1N HCl and brine. The organic phase is dried over Na₂SO₄ and concentrated. Compound VII (where a=single bond, X═CO, R₃=2-iodophenyl, R₄═H, R₅═OCH₃) is isolated by flash chromatography (petroleum ether—EtOAc, 1:1). White solid, yield: 61%. ¹H NMR (200 MHz, CDCl₃): mixture of epimers and rotamers δ=7.80-7.64 (m, 1H), 7.38-7.21 (m, 2H), 7.11-7.01 (m, 1H), 5.61 (d, 1H major), 5.52 (d, 1H minor), 4.78 (s, 1H minor), 4.54-4.25 (m, 4H), 4.08-3.98 (m, 1H), 3.38 (s, 3H minor), 3.32 (s, 3H major), 3.22-3.15 (m, 1H major), 2.91-2.59 (m, 1H minor). ¹³C NMR (50 MHz, CDCl₃): mixture of epimers and rotamers δ=171.4 (s, 1C), 170.6-170.3 (s, 1C), 142.2 (s, 1C), 139.2 (d, 1C), 130.8-130.6 (d, 2C), 128.5-128.2 (d, 1C), 128.0-127.6 (d, 1C), 95.8-95.2 (d, 1C), 70.9 (t, 1C), 65.8-65.2 (d, 1C), 56.2-55.4 (q, 1C), 51.2 (d, 1C), 47.5-46.5 (t, 1C).

### Example 10

### Synthesis of (5R/S,3aS,7aR)-7-(2-bromobenzoyl)-5-methoxy-hexahydro-2,4-dioxa-7-aza-inden-1-one [structure VII where a=single bond, X═CO, R₃=2-bromophenyl, R₄═H, R₅═OCH₃]

To a solution of VII (where a=single bond, X=bond, R₃═H, R₄═H, R₇═CH₃) (1 eq) and TEA (1.5 eq) in anhydrous CH₂Cl₂ (2.5 mL/mmol) a solution of 2-bromobenzoyl chloride (1.2 eq) in anhydrous CH₂Cl₂ (2.5 mL/mmol) is added at 0° C.

The mixture is allowed to reach room temperature and is left overnight stirring under N₂. Successively the mixture is washed with NaHCO₃, 1N HCl and brine. The organic phase is dried over Na₂SO₄ and concentrated. Compound VII (where a=single bond, X═CO, R₃=2-bromophenyl, R₄═H, R₅═OCH₃) is isolated by flash chromatography (petroleum ether—EtOAc, 1:1). White solid, yield: 79%. ¹H NMR (200 MHz, CDCl₃): mixture of epimers and rotamers δ=7.59-7.52 (m, 1H), 7.44-7.19 (m, 3H), 5.66 (d, 1H major), 5.55 (d, 1H minor), 4.78 (s, 1H minor), 4.57-4.28 (m, 4H), 4.13-4.09 (m, 1H), 3.41 (s, 3H minor), 3.34 (s, 3H major), 3.26-3.18 (m, 1H major), 2.92 (dd, 1H minor). ¹³C NMR (50 MHz, CDCl₃): mixture of epimers and rotamers δ=170.9 (s, 1C), 169.3-168.9 (s, 1C), 135.8 (s, 1C), 133.4-132.8 (d, 1C), 130.9-130.7 (d, 2C), 128.8 (d, 1C), 127.9-127.4 (d, 1C), 95.7-95.2 (d, 1C), 70.8 (t, 1C), 65.4-65.2 (d, 1C), 56.1-55.5 (q, 1C), 51.2-50.9 (d, 1C), 47.3-46.3 (t, 1C).

### Example 11

### Synthesis of (5R/S,3aS,7aR)-7-(4-chlorobenzenesulfonyl)-5-methoxy-hexahydro-2,4-dioxa-7-aza-inden-1-one [structure VII where a=single bond, X═SO₂, R₃=4-chlorophenyl, R₄═H, R₅═OCH₃]

To a solution of VII (where a=single bond, X=bond, R₃═H, R₄═H, R₅═OCH₃) (1 eq), TEA (2.5 eq) and DMAP (0.2 eq) in anhydrous CH₂Cl₂ (10 mL/mmol) 4-chlorobenzenesulfonyl chloride (2 eq) is added at 0° C. The mixture is allowed to reach room temperature and is left overnight stirring under N₂. Successively the mixture is washed with NaHCO₃, 1N HCl and brine. The organic phase is dried over Na₂SO₄ and concentrated. Compound VII (where a=single bond, X═SO₂, R₃=4-chlorophenyl, R₄═H, R₅═OCH₃) is isolated by flash chromatography (petroleum ether—EtOAc, 1:1). White solid, yield: 87%. ¹H NMR (200 MHz, CDCl₃): mixture of epimers δ=7.96 (d, 2H minor), 7.82 (d, 2H major), 7.58 (d, 2H minor), 7.45 (d, 2H major), 4.86 (d, 1H), 4.61 (s, 1H minor), 4.51-4.28 (m, 4H), 3.64 (d, 1H), 3.44 (s, 3H minor), 3.30 (s, 3H major), 2.99 (dd, 1H major), 2.57 (dd, 1H minor). ¹³C NMR (50 MHz, CDCl₃): mixture of epimers δ=171.2 (s, 1C), 139.3 (s, 1C), 137.9 (s, 1C), 129.3-129.2 (d, 2C), 129.0-128.4 (d, 2C), 94.7 (d, 1C), 70.8-70.7 (t, 1C), 64.9 (d, 1C), 55.6-55.4 (q, 1C), 54.6-54.2 (d, 1C), 44.8-44.3 (t, 1C).

### Example 12

### Synthesis of (5R/S,3aS,7aR)-7-benzyl-5-methoxy-hexahydro-2,4-dioxa-7-aza-inden-1-one [structure VII where a=single bond, X=bond, R₃═benzyl, R₄═H, R₅═OCH₃]

To a solution of VII (where a=single bond, X=bond, R₃═H, R₄═H, R₅═OCH₃) (1 eq) and benzaldehyde (1 eq) in THF (5 mL/mmol) NaBH(OAc)₃ (1.3 eq) is added in small portions. The mixture is left overnight stirring at room temperature, then is concentrated, diluted with EtOAc and washed with H₂O and brine. The organic phase is dried over Na₂SO₄ and concentrated. Compound VII (where a=single bond, X=bond, R₃═benzyl, R₄═H, R₅═OCH₃) is isolated by flash chromatography (petroleum ether—EtOAc, 2:1). White solid, yield: 45%. ¹H NMR (200 MHz, CDCl₃): mixture of epimers δ=7.45-7.29 (m, 5H), 4.68 (s, 1H), 4.49-4.26 (m, 2H), 4.24 (s, 2H), 4.11 (d, 1H), 3.56 (d, 1H major), 3.51 (d, 1H minor), 3.48 (s, 3H minor), 3.43 (s, 3H major), 2.94-2.77 (m, 2H major), 2.60-2.50 (m, 2H minor). ¹³C NMR (50 MHz, CDCl₃): mixture of epimers δ=173.8 (s, 1C), 136.9 (s, 1C), 129.1 (d, 2C), 128.3 (d, 1C), 127.6 (d, 1C), 127.4 (d, 1C), 99.5-97.2 (d, 1C), 70.7-70.4 (t, 1C), 66.4 (d, 1C), 58.3-57.3 (q, 1C), 57.8 (t, 1C), 55.7 (d, 1C), 51.4-50.3 (t, 1C).

### Example 13

### Synthesis of (5R/S,3aS,7aR)-7-(carbomethoxy-4-tolylmethyl)-5-methoxy-hexahydro-2,4-dioxa-7-aza-inden-1-one [structure VII where a=single bond, X=bond, R₃═—CH(tolyl)COOMe, R₄═H, R₅═OCH₃]

A solution of VII (where a=single bond, X=bond, R₃═H, R₄═H, R₅═OCH₃) (1 eq), p-tolylboronic acid (1 eq) and glyoxylic acid (1 eq) in EtOH (3.5 mL/mmol) is left overnight stirring at room temperature, then is concentrated. The crude acid VII (where a=single bond, X=bond, R₃═—CH(tolyl)COOH, R₄═H, R₅═OCH₃) was dissolved in MeOH/CH₂Cl₂ (5 mL/mmol-5 mL/mmol) and was added TMSCHN₂ (2M in Et₂O) dropwise. The mixture is left 2h stirring at room temperature successively is concentrated. Compound VII (where a=single bond, X=bond, R₃═—CH(tolyl)COOMe, R₄═H, R₅═OCH₃) is isolated by flash chromatography (petroleum ether—EtOAc, 3:2). White solid, yield: 65%. ¹H NMR (200 MHz, CDCl₃): mixture of epimers δ=7.48 (d, 2H), 7.16 (d, 2H), 5.16 (s, 1H), 4.68 (s, 1H), 4.47 (s, 1H), 4.15 (s, 2H), 3.67 (s, 3H), 3.48 (s, 3H), 3.42 (d, 1H), 2.99-2.81 (m, 2H), 2.32 (s, 3H).

### Example 14

### Synthesis of (5R/S,3aS,7aR)-5-Hydroxy-hexahydro-2,4-dioxa-7-aza-inden-1-one [structure VII where a=single bond, X=bond, R₃═H, R₄═H, R₅═OH]

Compound II (where X=bond, R₃═H, R₁ and R₈═—C(CH₃)₂—OCH₂—, R₂═COOMe, R₄═H, R₇═CH₃) (1 eq) is dissolved in HCl 6N (3.5 mL/mmol). The mixture is left for 2h at 80° C. under N₂. Successively, the mixture is concentrated and filtered on a weakly basic resin, giving compound VII (where a=single bond, X=bond, R₃═H, R₄═H, R₅═OH) as a yellow oil. ¹H NMR (200 MHz, D₂O): mixture of epimers δ=5.19 (s, 1H), 4.77-4.74 (m, 1H), 4.52-4.48 (m, 1H), 4.42-4.39 (m, 1H minor), 4.37-4.34 (m, 1H major), 4.25-4.23 (d, 1H major), 4.19-4.17 (d, 1H minor), 3.04-2.81 (m, 2H). ¹³C NMR (50 MHz, D₂O): mixture of epimers δ=170.7-170.2 (s, 1C), 86.6-86.9 (d, 1C), 72.4-70.5 (t, 1C), 64.5 (d, 1C), 52.3-51.5 (d, 1C), 43.3-43.0 (t, 1C).

### Example 15

### Synthesis of (5R/S,3aS,7aR)-5-hydroxy-7-carbobenzyloxy-hexahydro-2,4-dioxa-7-aza-inden-1-one [structure VII where a=single bond, X═CO, R₃═—OCH₂Ph, R₄═H, R₅═OH]

To a solution of VII (where a=single bond, X=bond, R₃═H, R₄═H, R₅═OH) (1 eq) and NaHCO₃ (2 eq) in H₂O-EtOAc (1.7 mL/mmol-2 mL/mmol) benzylchloroformate (1 eq) is added at 0° C. The mixture is allowed to reach room temperature and is left overnight stirring under N₂. Successively, the mixture is washed with 1N HCl and brine. The organic phase is dried over Na₂SO₄ and concentrated. Compound VII (where a=single bond, X═CO, R₃═—OCH₂Ph, R₄═H, R₅═OH) is isolated by flash chromatography (petroleum ether—EtOAc, 1:2). White solid, yield: 53%. ¹H NMR (200 MHz, CDCl₃): mixture of epimers and rotamers δ=7.31 (s, 5H), 5.15 (s, 2H major), 5.12 (s, 2H minor), 4.90-4.64 (m, 2H), 4.30-4.23 (m, 3H), 4.03-3.87 (m, 1H), 3.03-2.88 (m, 1H major), 2.75-2.56 (m, 1H minor). ¹³C NMR (50 MHz, CDCl₃): mixture of epimers and rotamers δ=172.8-172.5 (s, 1C), 156.3-155.8 (s, 1C), 135.7-135.5 (s, 1C), 128.5 (d, 1C), 128.3 (d, 1C), 128.1 (d, 1C), 127.9 (d, 1C), 127.7 (d, 1C), 91.8-91.4 (d, 1C minor), 88.9-88.5 (d, 1C major), 71.2-70.7 (t, 1C), 68.3 (t, 1C), 64.9-64.5 (d, 1C), 53.9 (d, 1C minor), 53.4-52.8 (d, 1C, major), 45.9-45.4 (t, 1C minor), 44.7-44.1 (t, 1C major).

### Example 16

### Synthesis of (5R/S,3aS,7aR)-5-hydroxy-7-fluorenylmethoxycarbonyl-hexahydro-2,4-dioxa-7-aza-inden-1-one [structure VII where a=single, X═CO, R₃═(9H-fluoren-9-yl)methoxy-, R₄═H, R₅═OH]

To a solution of VII (where a=single bond, X=bond, R₃═H, R₄═H, R₅═OH) (1 eq) and 2,6-lutidine (2.5 eq) in dioxane (20 mL/mmol) Fmoc-Cl (1.5 eq) is added at 0° C. The mixture is allowed to reach room temperature and is left overnight stirring under N₂. Successively, the mixture is concentrated, dissolved in EtOAc and washed with 5% citric acid and brine. The organic phase is dried over Na₂SO₄ and concentrated. Compound VII (where a=single bond, X═CO, R₃═(9H-fluoren-9-yl)methoxy-, R₄═H, R₅═OH) is isolated by flash chromatography (petroleum ether—EtOAc, 1:2). White solid, yield: 58%. ¹H NMR (200 MHz, DMSO): mixture of epimers and rotamers δ=7.68-7.61 (m, 2H), 7.58-7.45 (m, 2H), 7.32-7.14 (m, 4H), 5.11 (s, 1H major), 5.05 (d, 1H minor), 4.81 (d, 1H minor), 4.72-4.55 (m, 1H major), 4.47-4.23 (m, 6H), 4.00-3.81 (m, 1H), 2.97 (dd, 1H major), 2.79-2.42 (m, 1H minor). ¹³C NMR (50 MHz, DMSO): mixture of epimers and rotamers δ=171.9 (s, 1C), 155.4-154.7 (s, 1C), 143.0-142.9 (s, 2C), 140.4 (s, 2C), 127.1 (d, 2C), 126.5 (d, 2C), 124.5 (d, 2C), 119.3 (d, 2C), 91.0 (d, 1C minor), 88.1-87.8 (d, 1C major), 70.5-70.1 (t, 1C), 67.8 (t, 1C), 64.1-63.8 (d, 1C), 55.3-52.8 (d, 1C), 46.4 (d, 1C), 44.6-43.9 (t, 1C).

### Example 17

### Synthesis of (3aS,7aR)-7-benzoyl-3,3a,7,7a-tetrahydro-2,4-dioxa-7-aza-inden-1-one [structure VII where a=double bond, X═CO, R₃═Ph, R₄═H, R₅═H]

A mixture of VII (where a=single bond, X═CO, R₃═Ph, R₄═H, R₅═OCH₃) (1 eq) and p-toluenesulfonic acid (0.1 eq) in toluene (10 mL/mmol) is refluxed (110° C.) for 3 h in the presence of 4 Å molecular sieves. Successively, the mixture is filtered over NaHCO₃, and purified by flash chromatography (petroleum ether—EtOAc, 3:2), giving compound VII (where a=double bond, X═CO, R₃═Ph, R₄═H, R₅═H) as a white solid, yield: 20%. ¹H NMR (200 MHz, CDCl₃): mixture of rotamers δ=7.61-7.58 (m, 2H), 7.50-7.41 (m, 3H), 6.04 (s, 1H), 5.86 (s, 1H), 5.71 (s, 1H), 4.59-4.50 (m, 3H). ¹³C NMR (50 MHz, CDCl₃): mixture of rotamers δ=186.8 (s, 1C), 158.1 (s, 1C), 133.3 (s, 1C) 131. (d, 2C), 128.6 (d, 2C), 128.2 (d, 1C), 106.1 (d, 1C), 101.0 (d, 1C), 71.0 (t, 1C), 70.1 (d, 1C), 51.8 (d, 1C).

### Example 18

### Synthesis of (3aS,7aR)-7-carbobenzyloxy-3,3a,7,7a-tetrahydro-2,4-dioxa-7-aza-inden-1-one [structure VII where a=double bond, X═CO, R₃═-OCH₂Ph, R₄═H, R₅═H]

A mixture of II (where X═CO, R₃═—OCH₂Ph, R₁ and R₈═—C(CH₃)₂—OCH₂—, R₂═COOMe, R₄═H, R₇═CH₃) (1 eq) and p-toluenesulfonic acid (0.1 eq) in toluene (10 ml/mmol) is refluxed (110° C.) for 3 hours in the presence of 4 Å molecular sieves. Successively the mixture is filtered on NaHCO₃ and purified by flash chromatography (petroleum ether—EtOAc, 3:2) giving compound VII (where a=double bond, X═CO, R₃═—OCH₂Ph, R₄═H, R₅═H) as a white solid yield: 23%. ¹H NMR (200 MHz, CDCl₃): mixture of rotamers δ=7.37 (s, 5H minor), 7.34 (s, 5H major), 6.46 (d, 1H minor), 6.30 (d, 1H major), 6.02 (d, 1H minor), 5.89 (d, 1H major), 5.24 (d, 2H minor), 5.18 (d, 2H major), 5.09-4.47 (m, 2H), 4.40 (d, 2H major), 4.35 (d, 2H minor).

Examples 19-23 demonstrate that according to Scheme 5, when III and IV are reacted in a polar protic solvent under acid catalysis, according to step i, compound II is obtained, which in turn gives compound VIIIb through step iii:

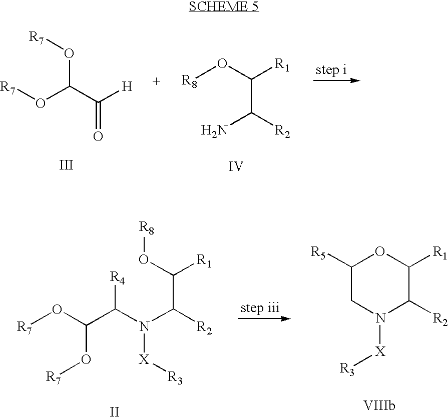

### Example 19

### Synthesis of methyl (2R,3S)-6-methoxy-2-methyl-morpholine-3-carboxylate [structure VIIIb where X=bond, R₁═CH₃, R₂═COOMe, R₃═H, R₅═OMe]

L-Thr(OTBDMS)—OMe (3.70 g, 14.9 mmol) (corresponding to compound IV where R₁═CH₃, R₂═COOMe, R₈=TBDMS) was dissolved in MeOH (45 mL), then 60% aqueous solution of dimethoxyacetaldehyde (2.59 g, 14.9 mmol) (corresponding to structure III where R₇═CH₃) and 10% Pd/C (329 mg) were successively added, and the resulting mixture was stirred overnight at room temperature under a hydrogen atmosphere. Then, the suspension was filtered on Celite and MeOH was removed under reduced pressure. The resulting mixture was partitioned between water and Et₂O. The combined organic layers were washed with brine, dried over Na₂SO₄ and concentrated under reduced pressure to yield compound II (where X=bond, R₁═CH₃, R₂═COOMe, R₃═H, R₇═CH₃, R₈=TBDMS) as a colourless oil (4.95 g, 99%). [α]²⁵_{D} −11.4 (c=1.1, CH₂Cl₂). ¹H NMR (400 MHz, CDCl₃) δ 4.53 (t, J=5.2 Hz, 1H), 4.18 (quintet, J=5.3 Hz, 1H), 3.73 (s, 3H), 3.37 (s, 6H), 2.94 (dd, J=12.2, 5.8 Hz, 1H), 2.73 (dd, J=12.2, 5.0 Hz, 1H). ¹³C NMR (50 MHz, CDCl₃) δ 171.9 (s), 102.9 (d), 69.1 (d), 66.8 (d), 54.4 (q), 53.6 (q), 52.0 (q), 48.9 (t), 25.7 (q, 3C), 20.8 (q), 17.9 (s), −4.2 (q), −5.1 (q).

SOCl₂ (511 μL, 7 mmol) was added dropwise, at 0° C., to 7 mL of MeOH. The resulting solution was used to dissolve compound II (where X=bond, R₁═CH₃, R₂═COOMe, R₃═H, R₄═H, R₇═CH₃, R₈=TBDMS) (600 mg, 1.79 mmol). The mixture was refluxed 4 h and successively concentrated under reduced pressure. The crude material was dissolved again in MeOH, eluted through Amberlist A21, and the solvent was evaporated to dryness to give VIIIb (where X=bond, R₁═CH₃, R₂═COOMe, R₃═H, R₅═OMe). ¹H NMR (400 MHz, CDCl₃) δ: 2 mixture of epimers δ4.47 (s, 0.4H), 4.40 (dd, J=8.8, 2.4 Hz, 0.6H), 3.90-3.85 (m, 0.4H), 3.74 and 3.73 (2s, 3H), 3.67-3.62 (m, 0.6H), 3.50 (s, 1.8H), 3.39 (s, 1.2H), 3.27 (d, J=9.4 Hz, 0.4H), 3.18 (d, J=9.4 Hz, 0.6H), 3.04 (dd, J=12.4, 2.4 Hz, 0.6H), 2.92-2.90 (m, 0.8H), 2.59 (dd, J=12.4, 8.8 Hz, 0.6H), 1.75-1.95 (bs, 1H), 1.25 (d, J=6.4 Hz, 1.8 Hz), 1.15 (d, J=6.0 Hz, 1.2H). ¹³C NMR (50 MHz, CDCl₃) mixture of epimers δ171.1 (s), 100.6 (d), 95.6 (d), 73.7 (d), 65.4 (d), 63.6 (d), 62.8 (d), 56.1 (q), 54.5 (q), 52.1 (q), 47.9 (t), 47.2 (t), 18.2 (q).

### Example 20

### Synthesis of methyl (2R,35)-6-methoxy-2-methyl-morpholine-4-benzyloxycarbonyl-3-carboxylate [structure VIIIb where X═CO, R₁═CH₃, R₂═COOMe, R₃═PhCH₂O—, R₅═OCH₃]

Compound VIIIb (where X=bond, R₁═CH₃, R₂═COOMe, R₃═H, R₅═OMe) was dissolved in 5 mL of 3:2 dioxane-water mixture, followed by addition of 98 mg (1.17 mmol) of NaHCO₃ and 84 μL (0.59 mmol) of Cbz-Cl. The mixture was left stirring for 16 h, then diluted with EtOAc and washed with 1N HCl and brine. The organic phase was dried over Na₂SO₄, and the solvent was evaporated, thus giving compound VIIIb (where X═CO, R₁═CH₃, R₂═COOMe, R₃═PhCH₂O—, R₅═OCH₃) as a colourless oil, 180 mg (61%). ¹H NMR (200 MHz, CDCl₃) δ=7.34 (m, 5H), 5.16 (s, 2H), 4.80-4.52 (m, 1H), 4.39-4.01 (m, 2H), 3.82-3.42 (m, 5H), 3.42 e 3.39 (2 s, 3H), 1.41 (d, J=6.6 Hz, 3H).

### Example 21

### Synthesis of methyl (3S,5S/R)-4-benzoyl-6-methoxy-morfolin-3-carboxylate [structure VIIIb where X═CO, R₁═H, R₂═COOMe, R₃═Ph, R₅═OCH₃]

To a solution of IV (where R₁═H, R₂═COOMe, R₈═H) (3.133 g, 13.4 mmol) in MeOH (70 mL) 1.94 g of 60% aqueous dimethoxyacetaldehyde solution was added (11.2 mmol) and 280 mg of 10% Pd/C. The mixture was left stirring under H₂ for 18 h at room temperature. The suspension was filtered over Celite, and the resulting solution was concentrated. After purification by flash chromatography (petroleum ether—EtOAc, 3:2) 3.18 g of II (where X=bond, R₁═H, R₂═COOMe, R₃═H, R₄═H, R₇═CH₃, R₈═H) (88%, R_{f} 0.62) were obtained as a pale yellow oil. [α]_{D}²⁵ −5.8 (c 1.6, CH₂Cl₂). ¹H NMR (200 MHz, CDCl₃) δ=4.41 (t, J=6.4 Hz, 1H), 3.80 (m, 2H), 3.68 (s, 3H, COOMe), 3.33 (s, 6H, Me acetal), 2.70 (dd, J=12.2, 6.4 Hz, 2H), 2.00 (bs, 1H, NH), 0.83 (s, 9H, tBu), 0.00 (s, 6H, SiMe₂). ¹³C NMR (50 MHz, CDCl₃) δ=173.2 (s, COOMe), 103.8 (d, CH(OMe)₂), 64.3 (t), 63.0 (d), 54.02 (q, 2C), 51.7 (q), 49.12 (t), 25.84 (q, 3C), 18.30 (s), −5.40 (q, 2C).

To a solution of II (where X=bond, R₁═H, R₂═COOMe, R₃═H, R₄═H, R₇═CH₃, R₈═H) (1.99 g, 6.20 mmol) in anhydrous THF and under a nitrogen atmosphere, DIPEA (1.27 mL, 7.44 mmol) was added. The mixture was cooled to 0° C., and benzoyl chloride (0.72 mL, 6.20 mmol) was dropwise added. The mixture was left stirring at room temperature for 18 h. The white suspension was diluted with a 1:1 H₂O/brine solution (10 mL), and successively treated with diethyl ether (2×15 mL) and dichloromethane (2×15 mL). The organic phases were combined, washed with brine and dried over Na₂SO₄. Compound II (where X═CO, R₁═H, R₂═COOMe, R₃=Ph, R₄═H, R₇═CH₃, R₈═H) (88%, R_{f} 0.62) was obtained after solvent evaporation in 80% yield. ¹H NMR (200 MHz, CDCl₃) mixture of rotamers δ=7.58-7.30 (m, 5H, Ph), 4.77-4.40 (m, 2H), 4.39-4.11 (m, 1H), 4.09-3.82 (m, 1H), 3.74 (s, 3H, COOMe), 3.60-3.38 (m, 4H), 3.29 (m, 2H), 3.17 (m, 2H), 0.87 (s, 9H, tBu), 0.06 (s, 6H, SiMe₂). ¹³C NMR (50 MHz, CDCl₃) mixture of rotamers δ=172.01 (s, COOMe) 169.46 (s, NCOPh), 136.02 (s, Cq Ph), 129.49 (d, Ph CH), 128.36 (d, Ph CH), 126.60 (d, Ph CH), 104.55 (d, CH acetal), 62.16 (d), 61.16 (t), 55.04 (q), 52.23 (q), 25.98 (q, tBu), 18.33 (s, tBu), −5.23 (q, SiMe₂).

To a solution of II (where X═CO, R₁═H, R₂═COOMe, R₃═Ph, R₄═H, R₇═CH₃, R₈═H) (3.17 g) in MeOH (185 mL) cooled to 0° C. SOCl₂ (0.271 mL, 3.71 mmol) was added. The mixture was left stirring at room temperature for 18 h, then solvent was evaporated. The crude yellow oil was dissolved in 40 mL of EtOAc, washed with 5% NaHCO₃, brine, and dried over Na₂SO₄. The resulting product, after solvent evaporation, was purified by flash chromatography (petroleum ether—EtOAc 1:1, R_{f} 0.52), thus giving 1.22 g of VIIIb (where X═CO, R₁═H, R₂═COOMe, R₃═Ph, R₅═OCH₃) as a yellow oil, yield: 87%.

¹H NMR (200 MHz, CDCl₃) mixture of epimers, mixture of rotamers δ=7.58-7.25 (m, 5H, Ph), 5.48 (s, 0.56H, CH acetal), 5.37 (s, 0.46H, CH acetal), 4.60-4.22 (m, 2H), 4.20-3.98 (m, 2H), 3.79 (s, 3H, COOMe), 3.68-3.20 (m, 4H). ¹³C NMR (50 MHz, CDCl₃) mixture of epimers, mixture of rotamers δ=171.2 (s), 169.0 (s), 134.3 (s, Ph), 129.9 (d, 2C, Ph), 128.4 (d, 2C, Ph), 126.8 (d, Ph), 99.3 e 94.9 (d, 1C, CH acetal), 64.2 (t), 58.7 (t), 56.2 (d), 52.7 (q), 51.8 (q). MS m/z (%) 279 (M⁺, 26), 248 (31), 218 (74), 105 (100), 77 (82).

### Example 22

### Synthesis of methyl (3S,5S/R)-4-[2-(9H-fluoren-9-ylmethoxycarbonylamino)-acetyl]-6-methoxy-morfolin-3-carboxylate [structure VIIIb where X═CO, R₁═H, R₂═COOMe, R₃═Fmoc-NHCH₂—, R₅═OCH₃]

To a solution of Fmoc-glycine (1.84 g, 6.20 mmol) in anhydrous THF (35 mL) N-methylmorpholine (0.75 mL, 6.80 mmol) was added, and, after cooling to 0° C., ethylchloroformate (0.59 mL, 6.20 mmol). The mixture was left stirring at 0° C. for 1 h, the a solution of amine II (where X=bond, R₁═H, R₂═COOMe, R₃═H, R₄═H, R₇═CH₃, R₈═H) (2.00 g, 6.20 mmol) in 10 mL of anhydrous THF was dropwise added. After stirring at room temperature for 18 h, the mixture was diluted with 40 mL of aqueous saturated NaHCO₃ solution and extracted with EtOAc (3×40 mL). The organic phases were combined and washed with aqueous 5% citric acid solution (2×50 mL), brine (1×50 mL) and dried over Na₂SO₄. The crude compound obtained after solvent evaporation was purified by flash chromatography (diethyl ether-petroleum ether 1:1, R_{f} 0.08), giving 1.961 g of II (where X═CO, R₁═H, R₂═COOMe, R₃═Fmoc-NHCH₂—, R₄═H, R₇═CH₃, R₈═H) as a yellow oil, yield: 53%. [α]_{D}²⁵ −27.8 (c=0.65, CH₂Cl₂). ¹H NMR (200 MHz, CDCl₃) mixture of rotamers δ=7.77-7.73 (m, 2H, H aromatic), 7.62-7.59 (m, 2H, H aromatic), 7.43-7.25 (m, 4H, H aromatic), 5.64-5.91 (br s, 1H, NH), 4.56 (t, J=4.5 Hz, 1H), 4.41-4.25 (m, 3H), 4.41-3.89 (m, 6H), 3.72 (s, 3H COOMe), 3.33-3.59 (m, [2H]), 3.44 e 3.42 (s, 6H, CH(OMe)₂), 0.88 (s, 9H, tBu), 0.06 (s, 6H, SiMe₂). ¹³C NMR (50 MHz, CDCl₃) mixture of rotamers δ=169.1 (s, 2C, COOMe e —CH₂CON), 156.0 (s, CO uretan), 143.8 (s, Fmoc), 141.1 (s, Fmoc), 127.6 (d, CH aromatic), 126.9 (d, CH aromatic), 125.1 (d, CH aromatic), 119.9 (d, CH aromatic), 103.5 (d, CH acetal), 67.1 (t), 63.1 (d), 61.2 (t), 55.5 e 54.9 (d, CH(OMe)₂), 52.3 ( ) 51.2 (t), 47.2 ( ) 43.0 (t), 25.9 (q, tBu), 18.3 (s, tBu), −5.3 (q, SiMe₂). MS m/z (%) 543 (31), 178 (96), 75 (100).

To a solution of II (where X═CO, R₁═H, R₂═COOMe, R₃═Fmoc-NHCH₂—, R₄═H, R₇═CH₃, R₈═H) (1.56 g, 2.60 mmol) in MeOH (80 mL) SOCl₂ (95 μL, 1.30 mmol) was dropwise added. The mixture was left reacting at room temperature for 18 h, then the solvent was evaporated and the product was dissolved in 30 mL of EtOAc, washed with 5% NaHCO₃ (1×20 mL) and brine (1×20 mL). After solvent evaporation the mixture was purified by flash chromatography (EtOAc-petroleum ether 1:1, R_{f} 0.22), resulting in 827 mg of VIIIb (where X═CO, R₁═H, R₂═COOMe, R₃═Fmoc-NHCH₂—, R₅═OCH₃) as a yellow oil. ¹H NMR (200 MHz, CDCl₃) mixture of epimers, mixture of rotamers δ=7.77-7.73 (m, 2H, H aromatic), 7.62-7.59 (m, 2H, H aromatic), 7.43-7.25 (m, 4H, H aromatic), 5.54-5.91 (bs, 1H, NH), 5.12 (s, 0.6H, CH acetal), 4.97 (s, 0.4H, CH acetal), 4.61 (s, 1H), 4.42-4.37 (m, 3H), 4.48-4.06 (m, 4H), 3.80 e 3.78 (s, 3H, COOMe), 3.70-3.51 (m, 1H), 3.46 (s, 1.5H), 3.35 (s, 1.5H), 3.3-3.0 (m, 1H). ¹³C NMR (50 MHz, CDCl₃) mixture of epimers, mixture of rotamers δ=169.4 (s), 168.6 (s), 156.1 (s), 143.8 (s, Fmoc), 141.2 (s, 2C, Fmoc), 126.9 (s, 2C, Fmoc), 127.6 (d, 2C, CH aromatic), 127.0 (d, 2C, CH aromatic), 125.1 (d, 2C, CH acetal), 67.3 (t), 64.23 (t) e 58.7 (t), 56.5 (d), 54.9 (q), 52.1 (q), 47.2 (d), 45.24 (t). ESI-MS (m/z): 493.27 (M⁺+2Na, 100), 477.36 (M⁺+Na, 8).

### Example 23

### Synthesis of methyl (3S,5S/R)-4-[2-benzyloxycarbonylamino-acetyl]-6-methoxy-morfolin-3-carboxylate [structure VIIIb where X═CO, R₁═H, R₂═COOMe, R₃═PhCH₂O—, R₅═OCH₃]

Compound II (where X=bond, R₁═H, R₂═COOMe, R₃═H, R₄═H, R₇═CH₃, R₈═H) (1.71 g, 5.31 mmol) was dissolved in a biphasic system consisting in EtOAc (12 mL) and H₂O (10 mL), then NaHCO₃ (1.71 g, 20.3 mmol) and, at 0° C., benzylchloroformate (1 eq) were added. The resulting mixture was left stirring at room temperature for 18 h, then it was diluted with 40 mL of EtOAc, treated with 5% citric acid (2×40 mL) and washed with brine (40 mL). The organic phase was dried over Na₂SO₄, and the crude product, obtained after solvent evaporation, was purified by flash chromatography (EtOAc-petroleum ether 1:2, R_{f} 0.59), giving 2.05 g of II (where X═CO, R₁═H, R₂═COOMe, R₃═PhCH₂O—, R₄═H, R₇═CH₃, R₈═H) as a yellow oil, yield: 84%. ¹H NMR (200 MHz, CDCl₃) mixture of rotamers δ=7.58-7.22 (m, 5H, Ph), 5.12-5.30 (m, 2H, CH₂Ph), 4.58-4.40 (m, 1H), 4.36 (pt, 1H), 4.00-4.21 (m, 2H), 3.70 (s, 3H, COOMe), 3.60-3.20 (m, 8H), 0.84 (s, 9H, tBu), 0.06 (s, 6H, SiMe₂). ¹³C NMR (50 MHz, CDCl₃) mixture of rotamers δ=169.85 (s, COOMe) 155.47 (s, NCOOBn), 136.35 e 136.00 (s, 1C, Ph), 128.05 (d, Ph), 127.96 (d, Ph), 127.85 (d, Ph), 104.39 e 104.13 (d, 1C, CH acetal), 67.52 (t, Ph-CH₂), 63.16 e 62.72 (d, 1C), 62.02 e 61.38 (t, 1C), 55.40 (q), 54.45 e 54.04 (q), 51.06 e 50.64 (d), 25.93 (q, tBu), 18.29 (s, tBu), −5.34 (q, SiMe₂). MS m/z (%) 456 (M⁺, 2), 125 (13), 91 (52), 75 (62), 57 (100).

Compound II (where X═CO, R₁═H, R₂═COOMe, R₃═PhCH₂O—, R₄═H, R₇═CH₃, R₈═H) (2.05 g, 4.50 mmol) was dissolved in 135 mL of MeOH, and after cooling to 0° C. SOCl₂ (0.160 mL, 2.19 mmol) was added. The mixture was left stirring at room temperature for 18 h, then, after solvent evaporation, the crude was dissolved in 60 mL of EtOAc. The organic phase was washed with 5% NaHCO₃ (3×50 mL), brine (1×60 mL), and dried over Na₂SO₄. After solvent evaporation, 1.27 g of VIIIb (where X═CO, R₁═H, R₂═COOMe, R₃═PhCH₂O—, R₅═OCH₃) were obtained as a yellow oil, yield: 91%. ¹H NMR (200 MHz, CDCl₃) mixture of epimers, mixture of rotamers δ=7.24-7.40 (m, 5H, Ph), 5.12-5.30 (m, 2H, CH₂Ph), 4.78-4.62 (m, 1H), 4.62-4.58 (m, 1H), 4.58-4.30 (m, 1H), 4.20-3.83 (m, 2H), 3.82-3.68 (m, 3H, COOMe), 3.51-3.28 (m, 3H, CHOMe), 3.19-2.87 (m, 1H). ¹³C NMR (50 MHz, CDCl₃) mixture of epimers, mixture of rotamers δ=170.0 (s, COOMe), 156.2 (s, NCOOBn), 136.1 (s, Ph), 128.4 (d, 2C, Ph), 128.1 (d, 2C, Ph), 127.9 (d, 1C, Ph), 99.8 e 95.0 (d, 1C, CH acetal), 67.7 (t, Ph-CH₂), 58.7 (t, CH₂O), 55.0 e 54.3 (q, 1C), 53.7 e 52.6 (q, 1C), 44.7 (t, 1C). MS m/z (%) 309 (M⁺, 22), 277 (15), 250 (58), 206 (44), 158 (52), 91 (100).

Examples 24-27 demonstrate that, according to Scheme 6, when III and IV are reacted in an apolar aprotic solvent in the presence of acid catalysis, and under refluxing conditions, according to step i, compound II is obtained, which in turn gives compound VIIIa through step iii:

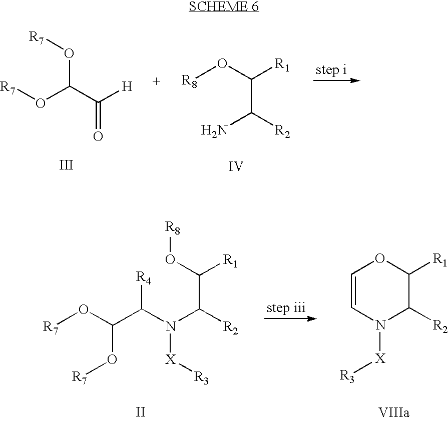

### Example 24

### Synthesis of methyl (2R,3S)-4-benzyloxycarbonyl-2-methyl-2,3-dihydro-[1,4]oxazin-3-carboxylate [structure VIIIa where X═CO, R₁═CH₃, R₂═COOMe, R₃═PhCH₂O—]

To a solution of VIIIb (where X═CO, R₁═CH₃, R₂═COOMe, R₃═PhCH₂O—, R₅═OCH₃) (165 mg, 0.51 mmol) in 5 mL of 19 mg (0.1 mmol) of p-toluenesulfonic acid were added. The mixture was refluxed for 2 h, then cooled and concentrated. The resulting oil was purified by flash chromatography (petroleum ether—EtOAc 3:1), giving 148 mg (91%) of compound VIIIa (where X═CO, R₁═CH₃, R₂═COOMe, R₃=PhCH₂O—). ¹H NMR (200 MHz, CDCl₃) mixture of rotamers, δ=7.41-7.22 (m, 5H), 6.43 (d, J=4.8 Hz, minor, 1H), 6.33 (d, J=4.8 Hz, major, 1H), 5.90 (d, J=4.8 Hz, minor, 1H), 5.79 (d, J=4.8 Hz, major, 1H), 5.31-5.18 (m, 2H), 4.98-4.51 (m, 2H), 3.78 (s, minor, 3H), 3.70 (s, minor, 3H), 1.30 (d, J=6.1 Hz, 3H).

### Example 25

### Synthesis of (3S)-3-Isobutyl-4-carbobenzyloxy-3,4-dihydro-2H-[1,4]oxazine [structure VIIIa where a=double bond, X═CO, R₁═H, R₂=isobutyl, R₃═—OCH₂Ph]

(S)-leucinol (200 μL, 1.55 mmol) (corresponding to compound IV, where R₁═H, R₂=isobutyl, R₈═H) is dissolved in MeOH (5 mL), and aqueous 60% dimethoxyacetaldehyde (270 μL, 1.55 mmol) (corresponding to compound III, where R₇═CH₃) and 10% Pd/C (22 mg) are successively added, and the resulting mixture is left stirring at room temperature for 18 h under a hydrogen atmosphere. The suspension is filtered over Celite and the solvent is evaporated, giving compound II (where X=bond, R₁═H, R₂=isobutyl, R₃═H, R₄═H, R₇═CH₃, R₈═H) as a colourless oil. ¹H NMR (200 MHz, CDCl₃) δ=4.62-4.20 (m, 2H), 3.95 (m, 1H), 3.57 (m, 1H), 3.45 and 3.41 (s, 6H), 3.16 (m, 1H), 2.77 (m, 1H), 2.03 (br, 2H), 1.63-1.18 (m, 3H), 0.93 (m, 6H).

Amine II (where X=bond, R₁═H, R₂=isobutyl, R₃═H, R₄═H, R₇═CH₃, R₈═H) (307 mg, 1.5 mmol) is dissolved in EtOAc (3 mL) and H₂O (3 mL), then NaHCO₃ (260 mg, 3 mmol) and, at 0° C., benzylchloroformate (130 μL, 1.5 mmol) are added. The mixture is stirred for 3 h at room temperature, then EtOAc is added. The mixture is treated with 1N HCl and the organic phase is washed with brine and dried over Na₂SO₄. After solvent evaporation, the crude is purified by flash chromatography (petroleum ether—EtOAc 1:2), giving 186 mg of II (where X═CO, R₁═H, R₂=isobutyl, R₃═PhCH₂O—, R₄═H, R₇═CH₃, R₈═H) as a pale yellow oil, yield: 34%. ¹H NMR (200 MHz, CDCl₃) 2:1 mixture of rotamers, δ=7.36 (s, 5H), 5.20 (m, 2H), 4.89 and 4.53 (m, 1H), 4.13 (m, 2H), 3.54-3.35 (m, 2H), 4.13 and 4.10 (s, 3H, major rotamer), 3.49 and 3.45 (s, 3H, minor rotamer), 3.10 (m, 1H), 1.53-1.16 (m, 3H), 0.85 (m, 6H).

A solution of II (where X═CO, R₁═H, R₂=isobutyl, R₃═PhCH₂O—, R₄═H, R₇═CH₃, R₈═H) (86 mg, 0.25 mmol) in toluene (5 mL) containing p-toluenesulfonic acid (4 mg, 0.02 mmol) is placed in a round-bottomed flask equipped with a dropping funnel containing 4 Å molecular sieves, and refluxed for 2 h, then the mixture is cooled and filtered over NaHCO₃. Toluene is evaporated, and crude product is purified by flash chromatography (petroleum ether—EtOAc 1:1) giving compound VIIIa (where a=double bond, X═CO, R₁═H, R₂=isobutyl, R₃═—OCH₂Ph) as an oil (70 mg, 99%). ¹H NMR (200 MHz, CDCl₃) δ: 2 mixture of rotamers, δ=7.36 (s, 5H), 6.25 (d, J=4.8 Hz, 0.4H, minor rotamer), 6.13 (d, J=4.0 Hz, 0.6H, major rotamer), 5.99 (d, J=4.8 Hz, 0.4H, minor rotamer), 5.86 (d, J=4.7 Hz, 0.6H, major rotamer), 5.18 (s, 2H), 4.33-3.78 (m, 3H), 1.62-1.36 (m, 3H), 0.98-0.86 (m, 6H).

### Example 26

### Synthesis of methyl (3S)-4-(9H-fluoren-9-ylmethoxycarbonyl)-2,3-dihydro-[1,4]oxazin-3-carboxylate [structure VIIIa where X═CO, R₁═H, R₂═COOMe, R₃═(9H-fluoren-9-yl)methoxy-]

L-serine methylester hydrochloride (1.00 g, 6.47 mmol) is dissolved in MeOH (20 mL), and triethylamine (902 μL, 6.47 mmol), aqueous 60% dimethoxyacetaldehyde (1.11 g, 6.47 mmol) and 10% Pd/C (90 mg) are successively added, and the resulting mixture is left stirring at room temperature for 18 h. The suspension is filtered over Celite and the solvent is evaporated. Then, the crude is evaporated by flash chromatography (CH₂Cl₂—MeOH 12:1, R_{f} 0.43), thus giving compound II (where X=bond, R₁═H, R₂═COOMe, R₃═H, R₄═H, R₇═CH₃, R₈═H) as a colourless oil (1.31 g, 98%). [α]²⁴_{D} −28.5 (c=1.0, CH₂Cl₂). ¹H NMR (400 MHz, CDCl₃) δ=4.44 (t, J=4.5 Hz, 1H), 3.77 (dd, J=11.2, 4.5 Hz, 1H), 3.74 (s, 3H), 3.59 (dd, J=12.5, 8.0 Hz, 1H), 3.40 (t, J=4.5 Hz, 1H), 3.36 (s, 6H), 2.84 (dd, J=12.5, 4.5 Hz, 1H), 2.65 (dd, J=12.5, 4.5 Hz, 1H), 2.39 (br, 1H). ¹³C NMR (50 MHz, CDCl₃) δ=173.1 (s), 103.5 (d), 62.7 (d), 62.5 (t), 53.9 (q), 53.1 (q), 52.0 (q), 49.1 (t). MS m/z 207 (M⁺, 26), 149 (13), 133 (18).

To a solution of II (where X=bond, R₁═H, R₂═COOMe, R₃═H, R₄═H, R₇═CH₃, R₈═H) (1.14 g, 5.5 mmol) in water-dioxane 2:1 (15 mL) NaHCO₃ (0.92 g, 11.0 mmol) is added, and the mixture is cooled to 0° C. Then, a solution of Fmoc-Cl (1.42 g, 5.5 mmol) in dioxane (15 mL) is slowly added over a period of 15 min, and the mixture is left stirring for 2.5 h. Successively, the mixture is diluted with EtOAc (40 mL) and treated with water (20 mL), and the organic phase is washed with 1M HCl, brine and dried over Na₂SO₄. After solvent evaporation, the crude is purified by flash chromatography (EtOAc-petroleum ether, R_{f} 0.53), thus giving compound II (where X═CO, R₁═H, R₂═COOMe, R₃═(9H-fluoren-9-yl)methoxy-, R₄═H, R₇═CH₃, R₈═H) as a colourless oil (2.29 g, 97%). [α]²²_{D} −31.6 (c=1.0, CH₂Cl₂). ¹H NMR (400 MHz, CDCl₃) mixture of rotamers 3:2, δ=7.76 (d, J=7.2 Hz, 2H), 7.60 (d, J=6.0 Hz, 1H), 7.55-7.53 (m, 1H), 7.42-7.39 (m, 2H), 7.35-7.29 (m, 2H), 4.76-4.69 (m, 2H), 4.61-4.47 (m, 2H), 4.24-4.21 (m, 1H), 3.97-3.94 (m, 1H), 3.86-3.80 (m, 1H), 3.69 and 3.61 (s, 3H), 3.69-3.59 (m, 1H), 3.49 and 3.43 (2s, 2.4H), 3.16 e 3.11 (2s, 3.6H), 3.21-3.11 (m, 0.4H), 2.97 (dd, J=15.2, 7.2 Hz, 0.6H). ¹³C NMR (50 MHz, CDCl₃) δ=170.0 (s), 156.6 (s), 143.4 (s, 2C), 141.2 (s, 2C), 127.6 (d, 2C), 127.1 e 126.9 (d, 2C), 124.6 e 124.5 (d, 2C), 119.9 (d, 2C), 103.3 e 103.0 (d), 67.7 e 66.6 (t), 62.9 e 62.1 (d), 60.7 e 60.2 (t), 55.6 (q), 54.7 (q), 52.2 (q), 49.1 e 48.9 (t), 47.3 e 47.0 (d). A solution of II (where X═CO, R₁═H, R₂═COOMe, R₃═(9H-fluoren-9-yl)methoxy-, R₄═H, R₇═CH₃, R₈═H) (1.1 g, 2.56 mmol) in toluene (25 mL) containing p-toluenesulfonic acid (49 mg, 0.26 mmol) is placed in a round-bottomed flask equipped with a dropping funnel containing 13 g of 4 Å molecular sieves, and refluxed for 1.75 h, then the mixture is cooled and filtered over NaHCO₃. Toluene is evaporated, and crude product is purified by flash chromatography (petroleum ether—EtOAc 3:1, R_{f} 0.55) giving compound VIIIa (where X═CO, R₁═H, R₂═COOMe, R₃₌(9H-fluoren-9-yl)methoxy-) as a white solid (795 mg, 85%). [α]²³_{D} +6.2 (c=1.0, CH₂Cl₂). ¹H NMR (400 MHz, CDCl₃) mixture of rotamers 3:2, δ=7.77 (t, J=8.0 Hz, 1H), 7.61 (t, J=8.0 Hz, 1H), 7.50 (m, 1H), 7.41 e 7.32 (m, 2H), 6.42 (d, J=5.2 Hz, 0.4H), 6.36 (d, J=5.2 Hz, 0.6H), 6.02 (d, J=5.2 Hz, 0.4H), 5.98 (d, J=5.2 Hz, 0.6H), 4.97 (s, 0.4H), 4.68 (d, J=11.2 Hz, 0.4H), 4.60-4.40 (m, 3.2H), 4.32 (t, J=7.2 Hz, 0.6H), 4.23 (t, J=7.2 Hz, 0.4H), 3.99 (dd, J=11.2, 3.2 Hz, 0.6H), 3.87 (dd, J=11.2, 3.2 Hz, 0.4H), 3.86 (s, 1.8H), 3.71 (s, 1.2H). ¹³C NMR (50 MHz, CDCl₃) δ=168.2 (s), 151.9 e 151.2 (s), 143.4 e 143.2 (s, 2C), 141.0 (s, 2C), 129.7 e 128.8 (d), 127.6 (d, 2C), 126.9 (d, 2C), 124.9 (d), 124.8 (d), 124.8 e 124.5 (d), 119.9 (d, 2C), 105.9 e 105.3 (d), 68.3 e 67.8 (t), 65.4 e 64.9 (t), 54.5 e 53.9 (d), 52.8 (q), 47.0 e 46.9 (d).

### Example 27

### Synthesis of methyl (3S)-4-benzyloxycarbonyl-2,3-dihydro-[1,4]oxazin-3-carboxylate [structure VIIIa where X═CO, R₁═H, R₂═COOMe, R₃═PhCH₂O—]

Amine II (where X=bond, R₁═H, R₂═COOMe, R₃═H, R₄═H, R₇═CH₃, R₈═H) (7.10 g, 34.3 mmol) is dissolved in a biphasic system consisting in EtOAc (75 mL) and H₂O (60 mL), then NaHCO₃ (5.46 g, 68.6 mmol) and, at 0° C., benzylchloroformate (4.8 mL, 33.6 mmol) are added. The mixture is stirred for 3 h at room temperature, then EtOAc is added. The mixture is treated with 1N HCl and the organic phase is washed with brine and dried over Na₂SO₄. After solvent evaporation, the crude is purified by flash chromatography (petroleum ether—EtOAc 2:3), giving 10.77 g of II (where X═CO, R₁═H, R₂═COOMe, R₃═PhCH₂O—, R₄═H, R₇═CH₃, R₈═H) as a pale yellow oil, yield: 92%. ¹H NMR (200 MHz, CDCl₃) (mixture of rotamers) δ=7.40-7.22 (m, 5H, Ph), 5.12-5.30 (m, 2H), 4.80-4.40 (m, 2H), 4.36 (m, 1H), 4.10-3.78 (m, 3H), 3.75-3.65 (m, 3H), 3.70-3.50 (m, 2H), 3.43 (d, 3H), 3.38-3.10 (m, 4H).

To a solution of compound II (dove X═CO, R₁═H, R₂═COOMe, R₃═PhCH₂O—, R₄═H, R₇═CH₃, R₈═H) (9.95 g, 29.1 mmol) in 300 mL of toluene 554 mg (2.9 mmol) of p-toluenesulfonic acid are added. The mixture is refluxed for 3 h, then cooled and filtered over NaHCO₃. After solvent evaporation, the resulting oil is purified by flash chromatography (petroleum ether—EtOAc 3:1), thus giving 6.34 g (78%) of compound VIIIa (where X═CO, R₁═H, R₂═COOMe, R₃═PhCH₂O—). ¹H NMR (200 MHz, CDCl₃) mixture of rotamers, δ=7.43-7.22 (m, 5H), 6.53 (d, J=4.6 Hz, minor, 1H), 6.35 (d, J=4.6 Hz, major, 1H), 6.01 (d, J=4.6 Hz, minor, 1H), 5.90 (d, J=4.6 Hz, major, 1H), 5.23-5.18 (m, 2H), 4.95 (s, major, 1H), 4.83 (s, minor, 1H), 4.66-4.53 (m, 1H), 3.97 (t, J=4.0 Hz major, 1H), 3.90 (t, J=4.0 Hz minor, 1H), 3.77 (s, major, 3H), 3.70 (s, minor, 3H).

Examples 28-31 demonstrate that compounds VII, having a=single bond and R₅═OH, can be transformed into the corresponding trichloroacetimidate derivatives as precursors for compounds of general formula VII having R₅=alkyl or Wang resin.

### Example 28

### Synthesis of (3aS,5R/S,7aR)-7-fluorenylmethoxycarbonyl-5-trichloroacetimido-hexahydro-2,4-dioxa-7-aza-inden-1-one [structure VII where a=single bond, X═CO, R₃═(9H-fluoren-9-yl)methoxy, R₄═H, R₅═—C(N)CCl₃]

To a solution of VII (where a=single bond, X═CO, R₃═(9H-fluoren-9-yl)methoxy, R₄═H, R₅═OH) (1 eq) in anhydrous CH₂Cl₂ (5 mL/mmol) trichloroacetonitrile (2 eq) is added dropwise and DBU (0.1 eq) at 0° C. The mixture is allowed to reach room temperature and is left 2h stirring under N₂. Successively the mixture is diluted with Et₂O and washed with H₂O and brine. The organic phase is dried over Na₂SO₄ and concentrated. Compound VII (where a=single bond, X═CO, R₃═(9H-fluoren-9-yl)methoxy, R₄═H, R₅═—C(N)CCl₃) is isolated by flash chromatography (petroleum ether—EtOAc, 1:2). White solid, yield: 38%. ¹H NMR (200 MHz, CDCl₃): mixture of epimers and rotamers δ=8.65 (d, 1H), 7.76 (d, 2H), 7.55 (d, 2H), 7.43-7.29 (m, 4H), 6.21 (d, 1H), 5.25 (d, 1H), 4.87-4.23 (m, 7H), 3.35-3.14 (m, 1H). ¹³C NMR (50 MHz, CDCl₃): mixture of epimers and rotamers δ=171.3 (s, 1C), 160.1 (s, 1C) 155.6 (s, 1C), 143.3 (s, 2C), 141.1 (s, 2C), 127.8 (d, 2C), 127.1 (d, 2C), 124.8 (d, 2C), 120.0 (d, 2C), 92.2 (d, 1C), 77.1 (s, 1C), 70.5 (t, 1C), 68.8 (t, 1C), 67.0-66.6 (d, 1C), 53.6-53.1 (d, 1C), 47.1 (d, 1C), 42.7-42.0 (t, 1C).

### Example 29

### Synthesis of (3aS,5R/S,7aR)-7-carbobenzyloxy-5-trichloroacetimido-hexahydro-2,4-dioxa-7-aza-inden-1-one [structure VII where a=single bond, X═CO, R₃═—OCH₂Ph, R₄═H, R₅═—C(N)CCl₃]

To a solution of VII (where a=single bond, X═CO, R₃═—OCH₂Ph, R₄═H, R₅═OH) (1 eq) in anhydrous CH₂Cl₂ (5 mL/mmol) trichloroacetonitrile (2 eq) is added dropwise and DBU (0.1 eq) at 0° C. The mixture is allowed to reach room temperature and is left 2h stirring under N₂. Successively the mixture is diluted with Et₂O and washed with H₂O and brine. The organic phase is dried over Na₂SO₄ and concentrated. Compound VII (where a=single bond, X═CO, R₃═—OCH₂Ph, R₄═H, R₅═—C(N)CCl₃) is isolated by flash chromatography (petroleum ether—EtOAc, 1:2). White solid, yield: 46%. ¹H NMR (200 MHz, CDCl₃): mixture of epimers and rotamers δ=8.65 (d, 1H), 7.33-7.31 (m, 5H), 6.18 (d, 1H), 5.24-4.99 (m, 3H major and minor), 4.73-4.67 (m, 1H), 4.42-4.24 (m, 3H major and minor), 3.29-3.11 (m, 1H). ¹³C NMR (50 MHz, CDCl₃): mixture of epimers and rotamers δ=171.4 (1C), 160.1 (1C), 155.5 (1C), 135.5 (1C), 128.5 (2C), 128.3 (1C), 128.0 (1C), 127.9 (1C), 92.4-92.1 (1C), 77.1 (s, 1C), 70.5 (1C), 68.4 (1C), 67.0-66.7 (1C), 53.6-53.2 (1C), 42.8-42.1 (1C).

### Example 30

### Synthesis of (3aS,5R/S,7aR)-5-benzyloxy-7-carbobenzyloxy-hexahydro-2,4-dioxa-7-aza-inden-1-one [structure VII where a=single bond, X═CO, R₃═—OCH₂Ph, R₄═H, R₅═—OCH₂Ph]

To a solution of VII (where a=single bond, X═CO, R₃═—OCH₂Ph, R₄═H, R₅═—C(N)CCl₃) (1 eq) and benzyl alcohol (1 eq) in anhydrous CH₂Cl₂-Cyclohexane (2.2 mL/mmol-4.4 mL/mmol) BF₃.2Et₂O (0.1 eq) is added dropwise at 0° C. The mixture is allowed to reach room temperature and is left 2h stirring under N₂. Successively the mixture is concentrated, dissolved in EtOAc and washed with NaHCO₃ and brine. The organic phase is dried over Na₂SO₄ and concentrated. Compound VII (where a=single bond, X═CO, R₃═—OCH₂Ph, R₄═H, R₅═—OCH₂Ph) is isolated by flash chromatography (petroleum ether—EtOAc, 1:1). White solid, yield: 93%. ¹H NMR (200 MHz, CDCl₃): mixture of epimers and rotamers δ=7.34-7.31 (m, 10H), 5.29-5.12 (m, 3H), 4.89-4.31 (m, 6H), 4.08-3.97 (m, 1H), 3.16-2.98 (m, 1H). ¹³C NMR (50 MHz, CDCl₃): mixture of epimers and rotamers δ=171.9-171.6 (1C), 155.8-155.0 (1C), 136.3-136.2 (1C), 135.6-135.4 (1C), 128.2 (2C), 127.9 (2C), 127.8 (2C), 127.7 (1C), 127.6 (1C), 127.4 (1C), 127.3 (1C), 93.1-92.7 (1C), 70.4-70.3 (1C), 69.1-69.0 (1C), 67.9-67.8 (1C), 65.0-64.6 (1C), 53.3-53.0 (1C), 43.8-43.1 (1C).

### Example 31

### Loading of (3aS,5R/S,7aR)-5-hydroxy-7-carbobenzyloxy-hexahydro-2,4-dioxa-7-aza-inden-1-one on Wang resin [structure VII where a=single bond, X═CO, R₃═—OCH₂Ph, R₄═H, R₅═—OCH₂-polystyrene]

To a suspension of Wang resin (44 mg, 1 mmol/g) in anhydrous dichloromethane (1 mL) compound VII (where a=single bond, X═CO, R₃═—OCH₂Ph, R₄═H, R₅═—OH) (56 mg, 0.1 mmol) was added, followed by slow addition of BF₃.Et₂O (3 μL, 0.02 mmol) at 0° C. The mixture was left shaking at room temperature for 2 h, then the solution was filtered and the resin washed thoroughly with dichloromethane and methanol. Resin substitution was determined by measuring the UV absorbance at 301 nm of the Fmoc removal with piperidine, according to the literature, resulting in a resin substitution of 0.2 mmol/g.

Examples 32-44 demonstrate that compounds of general formula VII react with amines or hydroxylamines to give compounds IX which can be further transformed into compounds of formulae X-XII when R₁₀═H, according to Scheme 7:

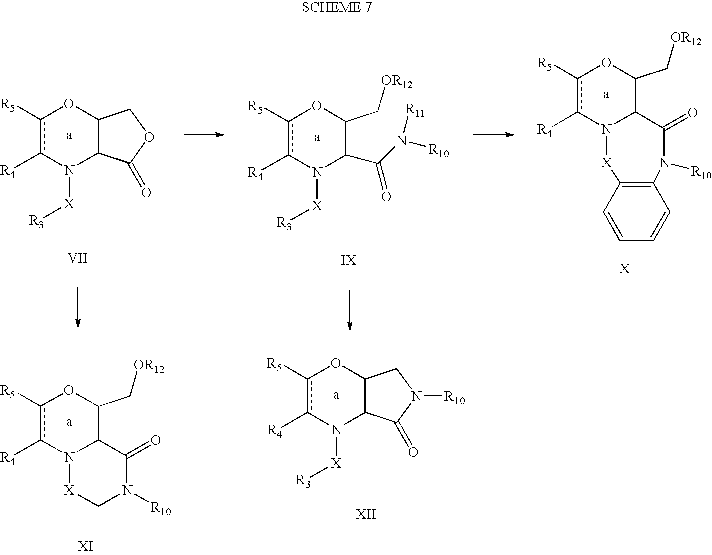

General procedure A for the preparation of compounds of general formula IX. To a solution of VII (1 eq) and 2-hydroxypyridine (5 eq) in anhydrous THF (20 mL/mmol) the selected amine (10 eq) is added. The mixture is left overnight stirring at room temperature under N₂, successively the mixture is washed with 1N HCl and brine. The organic phase is dried over Na₂SO₄, filtered and concentrated, and purified by flash chromatography (petroleum ether—EtOAc, 1:1), thus giving product IX.

### Example 32

### Synthesis of benzyl (5R/S,3aS,7aR)-4-benzoyl-2-hydroxymethyl-6-methoxy-morpholin-3-carboxamide [structure IX where a=single bond, X═CO, R₃═Ph, R₄═H, R₅═OCH₃, R₁₀═H, R₁₁=benzyl, R₁₂═H]

Compound VII (where a=single bond, X═CO, R₃═Ph, R₄═H, R₅═OCH₃) reacted with benzylamine according to general procedure A, thus giving product IX (where a=single bond, X═CO, R₃═Ph, R₄═H, R₅═OCH₃, R₁₀═H, R₁₁=benzyl, R₁₂═H) as a white solid, yield: 93%. ¹H NMR (400 MHz, CDCl₃): mixture of epimers and rotamers δ=7.50-7.28 (m, 10H), 7.01 (br, 1H), 5.15 (d, 1H), 4.70 (s, 1H), 4.49 (dq, 2H), 4.36-4.32 (m, 1H), 4.03 (dd, 1H), 3.84 (br, 1H), 3.71 (dd, 2H) 3.41 (s, 3H). ¹³C NMR (50 MHz, CDCl₃): mixture of epimers and rotamers δ=172.3 (s, 1C), 168.0 (s, 1C), 137.9 (s, 1C), 134.1 (s, 1C), 130.3 (d, 1C), 128.6 (d, 2C), 128.4 (d, 2C), 127.7 (d, 2C), 127.6 (d, 2C), 127.3 (d, 1C), 95.7 (d, 1C), 68.2 (d, 1C), 61.9 (t, 1C), 54.9 (q, 1C), 53.3 (d, 1C), 48.0 (t, 1C), 43.5 (t, 1C).

### Example 33

### Synthesis of benzyl (5R/S,3aS,7aR)-4-(2-iodobenzoyl)-2-hydroxymethyl-6-methoxy-morpholin-3-carboxamide [structure IX where a=single bond, X═CO, R₃═Ph, R₄═H, R₅═OCH₃, R₁₀═H, R₁₁=benzyl, R₁₂═H]

To a solution of VII (where a=single bond, X═CO, R₃=2-iodophenyl, R₄═H, R₅═OCH₃) (1 eq) in MeOH (0.35 mL/mmol) benzylamine (2 eq) is added. The mixture is left overnight stirring at room temperature, successively the mixture is washed with 1N HCl and brine. The organic phase is dried over Na₂SO₄, concentrated and purified by flash chromatography (petroleum ether—EtOAc, 1:1), thus giving product IX (where a=single bond, X═CO, R₃═Ph, R₄═H, R₅═OCH₃, R₁₀═H, R₁₁=benzyl, R₁₂═H) as a white solid, yield: 97%. ¹H NMR (200 MHz, CDCl₃): mix of epimers and rotamers δ=7.70 (d, 1H), 7.42-7.35 (m, 2H), 7.28 (s, 5H), 7.09-7.03 (m, 2H), 5.36 (d, 1H major), 5.18 (d, 1H minor), 4.60 (s, 1H), 4.51 (t, 2H), 4.32-4.25 (m, 1H), 4.01-3.83 (m, 2H), 3.63-3.38 (m, 1H), 3.31 (s, 3H) 3.26-3.19 (m, 1H). ¹³C NMR (50 MHz, CDCl₃): mix of epimers and rotamers δ=170.9-170.7 (s, 1C), 167.4-167.2 (s, 1C), 141.1-140.9 (s, 1C), 139.5-138.8 (d, 1C), 138.0 (s, 1C), 137.5 (s, 1C), 130.6 (d, 1C), 128.6 (d, 2C), 128.4 (d, 2C), 128.2 (d, 1C), 127.5 (d, 1C), 127.2-126.9 (d, 1C), 96.2-95.5 (d, 1C), 68.6-67.8 (d, 1C), 62.1-61.2 (t, 1C), 56.2-54.8 (q, 1C), 53.4-53.0 (d, 1C), 48.0-46.9 (t, 1C), 43.8-43.5 (t, 1C).

### Example 34

### Synthesis of allyl (5R/S,3aS,7aR)-4-benzoyl-2-hydroxymethyl-6-methoxy-morfolin-3-carboxamide [structure IX where a=single bond, X═CO, R₃═Ph, R₄═H, R₅═OCH₃, R₁₀═H, R₁₁=allyl, R₁₂═H]

Compound VII (where a=single bond, X═CO, R₃═Ph, R₄═H, R₅═OCH₃) reacted with allylamine according to general procedure A, thus giving product IX (where a=single bond, X═CO, R₃═Ph, R₄═H, R₅═OCH₃, R₁₀═H, R₁₁=allyl, R₁₂═H) as a white solid, yield: 97%. ¹H NMR (400 MHz, CDCl₃): mixture of epimers and rotamers δ=7.53-7.49 (m, 2H), 7.42-7.39 (m, 3H), 6.83 (br, 1H), 5.88-5.81 (m, 1H), 5.25-5.14 (m, 3H), 4.70 (s, 1H major), 4.61 (s, 1H minor), 4.33 (q, 1H), 4.03-3.75 (m, 7H) 3.75 (s), 3.41 (s, 3H). ¹³C NMR (50 MHz, CDCl₃): mixture of epimers and rotamers δ=172.2 (s, 1C), 168.0 (s, 1C), 134.2 (s, 1C), 133.6 (d, 1C), 130.3 (d, 2C), 128.4 (d, 2C) 127.7 (d, 1C), 116.4 (t, 1C), 95.7 (d, 1C), 68.1 (d, 1C), 61.8 (t, 1C), 54.9 (q, 1C), 53.2 (d, 1C), 48.0-47.6 (t, 1C), 41.9 (t, 1C).

### Example 35

### Synthesis of propargyl (5R/S,3aS,7aR)-4-benzoyl-2-hydroxymethyl-6-methoxy-morfolin-3-carboxamide [structure IX where a=single bond, X═CO, R₃═Ph, R₄═H, R₅═OCH₃, R₁₀═H, R₁₁═—CH₂(CH₂)₃CH₂—, R₁₂═H]

Compound VII (where a=single bond, X═CO, R₃═Ph, R₄═H, R₅═OCH₃) reacted with propargylamine according to general procedure A, thus giving product IX (where a=single bond, X═CO, R₃═Ph, R₄═H, R₅═OCH₃, R₁₀═H, R₁₁═—CH₂C≡CH, R₁₂═H) as a yellow solid, yield: 93%. ¹H NMR (400 MHz, CDCl₃): mixture of epimers and rotamers δ=7.56-7.52 (m, 2H), 7.41-7.38 (m, 3H), 7.15 (br, 1H), 5.18 (d, 1H), 4.69 (s, 1H), 4.61 (s, 1H), 4.34 (q, 1H), 4.14-4.04 (m, 2H), 4.00-3.96 (m, 1H), 3.84-3.69 (m, 3H), 3.50 (s, 3H minor), 3.40 (s, 3H major), 2.23 (s, 1H). ¹³C NMR (50 MHz, CDCl₃): mixture of epimers and rotamers δ=172.1 (s, 1C), 167.7 (s, 1C), 133.9 (s, 1C), 130.0 (d, 1C), 128.4-128.2 (d, 2C), 127.4-126.8 (d, 2C), 95.4 (d, 1C), 71.2 (t, 1C), 67.7 (d, 1C), 61.2 (s, 1C), 54.5 (q, 1C), 52.5 (d, 1C), 47.7 (t, 1C), 28.7 (t, 1C).

### Example 36

### Synthesis of piperidinyl (5R/S,3aS,7aR)-4-benzoyl-2-hydroxymethyl-6-methoxy-morfolin-3-carboxamide [structure IX where a=single bond, X═CO, R₃═Ph, R₄═H, R₅═OCH₃, R₁₀ and R₁₁═—CH₂(CH₂)₃CH₂—, R₁₂═H]

Compound VII (where X═CO, R₃═Ph, R₄═H, R₅═OCH₃) reacted with piperidine according to general procedure A, thus giving product IX (where a=single bond, X═CO, R₃═Ph, R₄═H, R₅═OCH₃, R₁₀ and R₁₁═—CH₂(CH₂)₃CH₂—, R₁₂═H) as a yellow solid, yield: 52%. ¹H NMR (400 MHz, CDCl₃): mixture of epimers and rotamers δ=7.56-7.52 (m, 2H), 7.41-7.38 (m, 3H), 5.56 (d, 1H), 4.69 (d, 1H), 4.31 (q, 1H), 4.02 (dd, 1H), 3.87-3.64 (m, 5H), 3.55-3.48 (m, 2H), 3.36 (s, 3H), 2.60-2.56 (m, 1H), 1.64 (m, 6H). ¹³C NMR (50 MHz, CDCl₃): mixture of epimers and rotamers δ=171.7 (s, 1C), 167.0 (s, 1C), 134.8 (s, 1C), 129.9 (d, 1C), 128.3 (d, 2C), 127.5 (d, 2C), 95.8 (d, 1C), 68.5 (d, 1C), 62.5 (t, 1C), 54.7 (q, 1C), 48.2 (d, 1C), 47.2 (t, 1C), 43.3 (t, 1C), 26.9 (t, 1C), 25.9 (t, 1C), 24.7 (t, 1C).

### Example 37

### Synthesis of cyclopropyl (5R/S,3aS,7aR)-4-benzoyl-2-hydroxymethyl-6-methoxy-morfolin-3-carboxamide [structure IX where a=single bond, X═CO, R₃═Ph, R₄═H, R₅═OCH₃, R₁₀═H, R₁₁=cyclopropyl, R₁₂═H]

Compound VII (where a=single bond, X═CO, R₃═Ph, R₄═H, R₅═OCH₃) reacted with cyclopropylamine according to general procedure A, thus giving product IX (where a=single bond, X═CO, R₃═Ph, R₄═H, R₅═OCH₃, R₁₀═H, R₁₁=cyclopropyl, R₁₂═H) as a white solid, yield: 55%. ¹H NMR (400 MHz, CDCl₃): mixture of epimers and rotamers δ=7.56-7.52 (m, 2H), 7.41-7.38 (m, 3H), 7.21 (br, 1H), 5.16 (d, 1H), 4.67 (d, 1H), 4.33 (q, 1H), 3.98 (dd, 1H), 3.87-3.82 (m, 1H), 3.67-3.64 (m, 2H), 3.37 (s, 3H), 2.78 (m, 1H), 0.88 (d, 2H), 0.49 (d, 2H). ¹³C NMR (50 MHz, CDCl₃): mixture of epimers and rotamers δ=172.2 (s, 1C), 169.5 (s, 1C), 134.3 (s, 1C), 130.3 (d, 1C), 128.4 (d, 2C), 127.6 (d, 2C), 95.7 (d, 1C), 68.1 (d, 1C), 61.7 (t, 1C), 54.9 (q, 1C), 52.9 (d, 1C), 48.1 (t, 1C), 22.7 (d, 1C), 6.6 (t, 1C), 6.4 (t, 1C).

### Example 38

### Synthesis of benzyl (5R/S,3aS,7aR)-4-(2-iodobenzoyl)-2-acetyloxymethyl-6-methoxy-morpholin-3-carboxamide [structure IX where a=single bond, X═CO, R₃═Ph, R₄═H, R₅═OCH₃, R₁₀═H, R₁₁=benzyl, R₁₂═COCH₃]

To a solution of IX (where a=single bond, X═CO, R₃═Ph, R₄═H, R₅═OCH₃, R₁₀═H, R₁₁=benzyl, R₁₂═H) (1 eq) in anhydrous CH₂Cl₂ (2 mL/mmol) Ac₂O (3 eq) and DMAP (0.1 eq) are added. The mixture is left overnight stirring under N₂. Successively, the mixture is washed with H₂O/ice and 1M KHSO₄. The organic phase is dried over Na₂SO₄ and concentrated, giving Compound IX (where a=single bond, X═CO, R₃═Ph, R₄═H, R₅═OCH₃, R₁₀═H, R₁₁=benzyl, R₁₂═COCH₃) after flash chromatography (petroleum ether—EtOAc, 1:1). Colourless oil, yield: 95%. ¹H NMR (200 MHz, CDCl₃): mixture of epimers and rotamers δ=7.71 (d, 1H), 7.44-7.36 (m, 2H), 7.30 (s, 5H), 7.14-7.04 (m, 1H), 6.85 (br, 1H), 5.32 (d, 1H major), 5.21 (d, 1H minor), 4.75-4.33 (m, 6H), 3.55-3.40 (m, 1H), 3.34 (s, 3H) 3.27-3.20 (m, 1H), 2.08 (s, 3H). ¹³C NMR (50 MHz, CDCl₃): mixture of epimers and rotamers δ=170.7-170.3 (s, 1C), 166.2 (s, 1C), 140.2 (s, 1C), 139.0-138.7 (d, 1C), 137.6 (s, 2C), 130.6 (d, 1C), 128.6 (d, 2C), 128.4 (d, 2C), 128.0 (d, 2C), 127.5-127.2 (d, 1C), 95.3 (d, 1C), 66.9 (d, 1C), 64.3 (t, 1C), 54.6 (q, 1C), 52.6-52.4 (d, 1C), 46.7 (t, 1C), 43.7 (t, 1C), 20.9 (q, 1C).

### Example 39

### Synthesis of allyl (5R/S,3aS,7aR)-4-benzoyl-2-acryloyloxy-6-methoxy-morfolin-3-carboxamide [structure IX where a=single bond, X═CO, R₃═Ph, R₄═H, R₅═OCH₃, R₁₀═H, R₁₁=allyl, R₁₂═—C(O)CH═CH₂]

To a solution of IX (where a=single bond, X═CO, R₃═Ph, R₄═H, R₅═OCH₃, R₁₀═H, R₁₁=allyl, R₁₂═H) (1 eq) and TEA (1 eq) in anhydrous CH₂Cl₂ (5 mL/mmol) acryloyl chloride (1.1 eq) is added at 0° C. The mixture is allowed to reach room temperature and left vernight stirring, then a saturated NaHCO₃ solution is added. The organic phase is washed with 1N HCl, a saturated NaHCO₃ solution and brine, and successively dried over Na₂SO₄, filtered and concentrated. Compound IX (where a=single bond, X═CO, R₃═Ph, R₄═H, R₅═OCH₃, R₁₀═H, R₁₁=allyl, R₁₂═—C(O)CH═CH₂) is obtained after flash chromatography (petroleum ether—EtOAc, 1:1) as a colourless oil, yield: 78%.

¹H NMR (400 MHz, CDCl₃): mixture of epimers and rotamers δ=7.56-7.55 (m, 2H), 7.46-7.40 (m, 3H), 6.68 (br, 1H), 6.45 (d, 1H), 6.16 (dd, 1H), 5.87 (d, 1H), 5.83-5.79 (m, 1H), 5.19-5.11 (m, 3H), 4.70-4.62 (m, 2H), 4.55-4.41 (m, 2H), 3.92-3.87 (m, 2H), 3.77 (d, 1H), 3.62 (d, 1H), 3.41 (s, 3H). ¹³C NMR (50 MHz, CDCl₃): mixture of epimers and rotamers δ=172.0 (s, 1C), 167.0 (s, 1C), 165.3 (s, 1C), 137.1 (d, 1C), 133.4 (d, 1C), 133.6 (s, 1C), 131.1 (t, 1C), 130.2 (d, 1C), 128.1 (d, 2C), 127.7 (d, 2C), 115.9 (t, 1C), 95.1 (d, 1C), 66.1 (d, 1C), 63.8 (t, 1C), 54.4 (q, 1C), 52.6 (d, 1C), 47.2 (t, 1C), 41.2 (t, 1C).

### Example 40

### Synthesis of (5R/S,3aS,7aR)-2-allyloxymethyl-4-benzoyl-6-methoxy-morpholine-3-carboxylic acid allyl-prop-2-ynyl-amide [structure IX where a=single bond, X═CO, R₃═Ph, R₄═H, R₅═OCH₃, R₁₀=propargyl, R₁₁=allyl, R₁₂=allyl]

To a solution of IX (where a=single bond, X═CO, R₃═Ph, R₄═H, R₅═OCH₃, R₁₀=propargyl, R₁₁═H, R₁₂═H) (1 eq) in anhydrous THF (10 ml/mmol) TBAI (0.01 eq) and allyl bromide (1 eq) were added. Then NaH (60% suspension in mineral oil; 20 mg, 3 eq) was added at 0° C. The mixture was allowed to reach room temperature and left overnight stirring, then washed with ice/water and extracted with EtOAc. The organic phase is dried over Na₂SO₄, filtered and concentrated. Compound IX (where a=single bond, X═CO, R₃═Ph, R₄═H, R₅═OCH₃, R₁₀=propargyl, R₁₁=allyl, R₁₂=allyl) is obtained after flash chromatography (petroleum ether—EtOAc, 1:1) as a colourless oil, yield: 88%.

¹H NMR (400 MHz, CDCl₃): mix of epimers and rotamers δ=7.50-7.38 (m, 5H), 5.96-5.75 (m, 2H), 5.62 (d, 1H major), 5.50 (d, 1H minor), 5.30-5.15 (m, 4H), 4.66 (m, 1H), 4.53-4.42 (m, 2H), 4.24-3.92 (m, 6H), 3.72-3.57 (m, 3H), 3.37 (s, 3H, minor), 3.35 (s, 3H, major), 2.28 (s, 1H minor), 2.20 (s, 1H major). ¹³C NMR (50 MHz, CDCl₃): mix of epimers and rotamers δ=171.4 (s, 1C), 168.0-167.5 (s, 1C), 134.9 (s, 1C), 134.4-134.2 (d, 1C), 133.0-132.1 (d, 1C), 129.9 (d, 1C), 128.3 (d, 2C), 127.5 (d, 2C), 118.4-118.0 (t, 1C), 117.2-116.5 (t, 1C), 95.6 (d, 1C), 73.0-72.4 (t, 1C), 69.9-69.8 (t, 1C), 67.5-67.0 (d, 1C), 54.7 (q, 1C), 50.1-49.8 (t, 1C), 48.7-48.5 (d, 1C), 47.7-47.4 (t, 1C), 36.9 (s, 1C) 33.8 (t, 1C).

### Example 41

### Synthesis of (2R/S,4aR,7aR)-4-Benzoyl-6-benzyl-2-methoxy-hexahydro-pyrrolo[3,4-b][1,4]oxazin-5-one [structure XII where a=single bond, X═CO, R₃═Ph, R₄═H, R₅═OCH₃, R₁₁=benzyl]

To a solution of IX (where a=single bond, X═CO, R₃═Ph, R₄═H, R₅═OCH₃, R₁₀=benzyl, R₁₁═H, R₁₂═H) (1 eq) in anhydrous toluene (10 mL/mmol), triphenylphosphine (2 eq) was added, then diisopropyl azodicarboxylate (2 eq) was added dropwise. The resulting yellow solution was left overnight stirring at room temperature, and successively the mixture is concentrated. Compound XII (where a=single bond, X═CO, R₃═Ph, R₄═H, R₅═OCH₃, R₁₁=benzyl) is isolated by flash chromatography (petroleum ether—EtOAc, 1:1). Colourless oil, yield: 45%. ¹H NMR (200 MHz, CDCl₃): mixture of epimers and rotamers δ=7.50 (d, 1H), 7.56-7.62 (m, 1H), 7.40-7.20 (m, 8H major and minor), 5.71 (d, 1H major), 5.50 (d, 1H minor), 5.18 (d, 1H minor), 4.75-4.05 (m, 6H), 3.57-3.50 (m, 1H), 3.43 (s, 3H minor), 3.38 (s, 3H major), 3.33 (s, 3H minor), 3.31-3.08 (m, 1H). ¹³C NMR (50 MHz, CDCl₃): mixture of epimers and rotamers δ=172.1 (s, 1C), 156.2 (s, 1C), 140.6-140.5 (s, 1C), 134.6-134.5 (s, 1C), 130.2-130.0.0 (d, 1C), 128.6-128.4 (s, 2C), 128.3-128.2 (d, 2C), 127.6-127.5 (d, 2C), 127.5-127.4 (d, 2C), 126.4-126.3 (d, 1C), 96.2-95.5 (d, 1C), 72.0-71.7 (t, 1C), 66.4-65.9 (d, 1C), 55.4-55.1 (q, 1C), 51.6 (d, 1C), 50.9-50.8 (t, 1C), 47.1-46.7 (t, 1C).

### Example 42

### Synthesis of (2R/S,4aR,7aR)-4-Benzoyl-6-cyclopropyl-2-methoxy-hexahydro-pyrrolo[3,4-b][1,4]oxazin-5-one [structure XII where a=single bond, X═CO, R₃═Ph, R₄═H, R₅═OCH₃, R₁₁=cyclopropyl]

To a solution of IX (where a=single bond, X═CO, R₃═Ph, R₄═H, R₅═OCH₃, R₁₀=cyclopropyl, R₁₁═H, R₁₂═H) (1 eq) in anhydrous toluene (10 mL/mmol), triphenylphosphine (1 eq) were added. To this stirred solution diisopropyl azodicarboxylate (1 eq) was added dropwise. The resulting yellow solution is left overnight stirring at room temperature, successively the mixture is concentrated. Compound XII (where a=single bond, X═CO, R₃═Ph, R₄═H, R₅═OCH₃, R₁₁=cyclopropyl) is isolated by flash chromatography (petroleum ether—EtOAc, 1:1). Yellow solid, yield: 58%. ¹H NMR (200 MHz, CDCl₃): mixture of epimers and rotamers δ=7.65-7.29 (m, 5H major and minor), 5.7 (d, 1H minor), 5.60 (d, 1H major), 4.74-4.07 (m, 6H), 3.42 (s, 3H minor), 3.36 (s, 3H major), 3.30-3.01 (m, 1H), 0.73-0.58 (m, 4H).

### Example 43

### Synthesis of (1S,3R/S,10R)-8-Benzyl-1-hydroxymethyl-3-methoxy-hexahydro-pyrazino[2,1-c][1,4]oxazine-6,9-dione [structure XI where a=single bond, X═CO, R₄═H, R₅═OCH₃, R₁₁=benzyl, R₁₂═H]

To a solution of VII (where a=single bond, X═CO, R₃═—CH₂Br, R₄═H, R₅═OCH₃) (1 eq) in MeOH (0.35 mL/mmol) benzylamine (2 eq) is added. The mixture is left overnight stirring at room temperature, successively the mixture is washed with 1N HCl and brine. The organic phase is dried over Na₂SO₄, concentrated and purified by flash chromatography (EtOAc), thus giving product XI (where a=single bond, X═CO, R₄═H, R₅═OCH₃, R₁₁=benzyl, R₁₂═H) as a white solid, yield: 59%. ¹H NMR (200 MHz, CDCl₃): mixture of epimers and rotamers δ=7.38-7.31 (m, 5H), 4.84 (t, 1H), 4.73 (d, 1H), 4.59 (d, 1H), 4.51-4.32 (m, 3H), 3.98-3.77 (m, 4H), 3.52 (s, 3H minor), 3.48 (s, 3H major), 2.89 (dd, 1H). ¹³C NMR (50 MHz, CDCl₃): mixture of epimers and rotamers δ=162.7-162.9 (s, 2C), 134.6 (s, 1C), 128.8 (d, 2C), 128.4-128.2 (s, 2C), 128.1 (s, 1C), 94.9 (d, 1C), 72.7 (d, 1C), 61.7 (t, 1C), 56.2 (q, 1C), 55.7 (d, 1C), 49.3 (t, 1C), 49.0 (t, 1C), 45.2 (t, 1C).

### Example 44

### Synthesis of (1S,3R/S,11aR)-10-Benzyl-1-hydroxymethyl-3-methoxy-1,3,4,11a-tetrahydro-10H-2-oxa-4a,10-diaza-dibenzo[a,d]cycloheptene-5,11-dione [structure X where a=single bond, X═CO, R₄═H, R₅═OCH₃, R₁₁=benzyl, R₁₂=acetyl]

A solution of IX (where a=single bond, X═CO, R₃=2-iodophenyl, R₄═H, R₅═OCH₃, R₁₀=benzyl, R₁₁═H, R₁₂=acetyl) (1 eq) in anhydrous DMSO (10 mL/mmol) was added under argon to a solution of CsOAc (10 eq) and CuI (2 eq) in anhydrous toluene. The mixture is left overnight stirring at 90° C., successively the mixture is diluted with Et₂O washed with ammoniacal NaCl. The organic phase is dried over Na₂SO₄, concentrated and purified by flash chromatography (petroleum ether—EtOAc, 1:1), thus giving product X (where a=single bond, X═CO, R₄═H, R₅═OCH₃, R₁₁=benzyl, R₁₂=acetyl) as a white solid, yield: 56%. ¹H NMR (200 MHz, CDCl₃): mixture of epimers and rotamers δ=7.45-7.36 (m, 2H), 7.30-7.25 (m, 5H), 7.14-7.07 (m, 1H), 6.92-6.84 (m, 1H), 5.32-5.29 (m, 1H), 5.10-5.04 (d, 1H), 4.75-4.32 (m, 5H), 3.78-3.54 (m, 1H), 3.41 (s, 3H major), 3.34 (s, 3H minor), 3.26-3.19 (m, 1H), 2.08 (s, 3H major), 2.05 (s, 3H minor). ¹³C NMR (50 MHz, CDCl₃): mixture of epimers and rotamers δ=171.9 (s, 1C), 170.6-170.0 (s, 1C), 167.0-166.0 (s, 1C), 139.8 (s, 1C), 138.4 (d, 1C), 137.8-137.1 (s, 1C), 133.6 (s, 1C), 130.3-130.0 (d, 1C), 128.4-128.3 (d, 2C), 128.2-128.1 (d, 2C), 128.0 (d, 1C), 127.5-127.4 (d, 1C), 127.0 (d, 1C), 95.1-94.9 (d, 1C), 66.6-65.9 (d, 1C), 63.9-63.6 (t, 1C), 54.3 (q, 1C), 52.4-52.1 (d, 1C), 47.1-46.3 (t, 1C), 43.4-42.8 (t, 1C), 20.6-20.5 (q, 1C).

Examples 45-48 demonstrate that compounds of general formula VIIIa react with a carbenoid species, preferably generated by Et₂Zn with CH₂I₂, or by alkyl diazoacetate with Cu(OTf)₂, to give compounds XIII, according to Scheme 8:

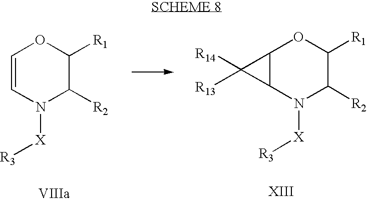

### Example 45

### Synthesis of methyl (1R,4S,6S)-2-Oxa-5-aza-bicyclo[4.1.0]heptane-4,5-dicarboxylic acid 5-benzyl ester 4-methyl ester [structure XIII where X═CO, R₁═H, R₂═COOMe, R₃═PhCH₂O—, R₁₃═R₁₄═H]

Compound VIIIa (where X═CO, R₁═H, R₂═COOMe, R₃═PhCH₂O—) (3.28 g, 11.7 mmol) is dissolved in 35 mL of anhydrous dichloromethane under a nitrogen atmosphere. The solution is cooled to −20° C., and a 1M solution of Et₂Zn in hexane is dropwise added. The mixture is stirred for 2 h at the same temperature, and for 21 h at room temperature, then successively treated with 10 mL of 5% NaHCO₃ and 5% citric acid (30 mL). The organic phase is washed with brine and dried over Na₂SO₄. After solvent evaporation, the crude compound is purified by flash chromatography (petroleum ether—EtOAc 3:1) giving XIII (where X═CO, R₁═H, R₂═COOMe, R₃═PhCH₂O—, R₁₃═R₁₄═H) as a colourless oil (2.24 g, 65%). ¹H NMR (400 MHz, CDCl₃) mixture of rotamers, δ=7.41-7.27 (m, 5H), 5.38-5.02 (m, 2H), 4.47 (t, J=6.8 Hz, major, 1H), 4.38 (t, J=6.8 Hz, minor, 1H), 3.98 (m, 1H), 3.80-3.59 (m, 5H), 2.85-2.81 (m, 1H), 0.98 (q, J=6.8 Hz, minor, 1H), 0.90 (q, J=6.8 Hz, major, 1H), 0.85-0.79 (m, 1H).
General procedure B: cyclopropanation with Cu(OTf)₂ and (S,S)-2,2′-isopropylidene-bis(4-tert-butyl-2-oxazoline). To a solution of dihydroxazine VIIIa (626 mg, 2.24 mmol) in dry CH₂Cl₂ (4 mL) cooled in an ice-salt bath were added Cu(OTf)₂ (16 mg, 0.045 mmol), (S,S)-2,2′-Isopropylidene-bis(4-tert-butyl-2-oxazoline) (16 mg, 0.056 mmol) and phenylhydrazine (4.4 μL, 0.045 mmol). After 30 min, a 1.2 M solution of diazoacetate in dry CH₂Cl₂ was added (quantity and time according to Table 1). During the addition the volume was maintained constant, expelling CH₂Cl₂ by passing nitrogen through the flask. The reaction was then gently warmed to room temperature and stirred 16 h. The mixture was concentrated under reduced pressure and the residue was purified by flash column chromatography (Hexanes-EtOAc 3:1) to yield the cyclopropanated products.

### Example 46

### Synthesis of (1R,4S,6S,7S)-2-Oxa-5-aza-bicyclo[4.1.0]heptane-4,5,7-tricarboxylic acid 5-benzyl ester 7-ethyl ester 4-methyl ester [structure XIII where X═CO, R₁═H, R₂═COOMe, R₃═—OCH₂Ph, R₁₃═H, R₁₄═COOEt]

Compound VIIIa (where X═CO, R₁═H, R₂═COOMe, R₃═PhCH₂O—) was treated according to general procedure B using 4.5 equivalents of ethyl-diazoacetate (time of addition 6 h) to yield XIII (where X═CO, R₁═H, R₂═COOMe, R₃═—OCH₂Ph, R₁₃═H, R₁₄═COOEt) (53%). [α]²⁵_{D} 5.5 (c=1.24, CHCl₃). ¹H NMR (400 MHz, CDCl₃) δ: 2 Mixture of rotamers δ7.35-7.28 (m, 5H), 5.29-5.11 (m, 2H), 4.20 (dd, J=13.6, 3.2 Hz, 1H), 4.20-4.05 (m, 3H), 3.84 (dd, J=11.6, 3.6 Hz, 0.6H), 3.80 (dd, J=11.6, 3.6 Hz, 0.4H), 3.74 (s, 1.8H), 3.63 (s, 1.2H), 3.53 (dd, J=7.2, 3.6 Hz, 0.4H), 3.49 (dd, J=7.2, 3.6 Hz, 0.6H), 2.38 (dd, J=3.6, 2.4 Hz, 0.6H), 2.29 (dd, J=3.6, 2.4 Hz, 0.4H), 1.26 and 1.21 (2t, J=7.2 Hz, 3H). ¹³C NMR (50 MHz, CDCl₃) Mixture of rotamers δ170.5 and 170.0 (s), 169.9 and 169.8 (s), 156.1 and 155.4 (s), 135.8 and 135.5 (s), 128.2-127.2 (d, 5C), 67.7 and 67.6 (t), 65.9 and 65.5 (t), 60.6 (t), 58.1 and 57.8(d), 55.4 and 54.9 (d), 52.6 (q), 35.3 and 35.2 (d), 27.5 and 27.3 (d), 14.2 (q). MS m/z 363 (M+, 1.2), 246 (15.2), 228 (17.4), 91 (100).

### Example 47

### Synthesis of (1R,4S,6S,7S)-2-Oxa-5-aza-bicyclo[4.1.0]heptane-4,5,7-tricarboxylic acid 5-benzyl ester 7-tert-butyl ester 4-methyl ester [structure XIII where X═CO, R₁═H, R₂═COOMe, R₃═—OCH₂Ph, R₁₃═H, R₁₄═COOt-Bu]

626 mg (2.24 mmol) of compound VIIIa (where X═CO, R₁═H, R₂═COOMe, R₃═PhCH₂O—) were treated according to general procedure B using 4.5 equivalents of tert-butyl-diazoacetate (time of addition 6 h) to yield 447 mg (51%) of structure XIII (where X═CO, R₁═H, R₂═COOMe, R₃═—OCH₂Ph, R₁₃═H, R₁₄═COOt-Bu). [α]²⁶_{D} 14.1 (c=0.6, CHCl₃). ¹H NMR (400 MHz, CDCl₃) 2:1 Mixture of rotamers δ7.37-7.27 (m, 5H), 5.31-5.12 (m, 2H), 4.25 (dd, J=14.0, 2.8 Hz, 1H), 4.13 (d, J=12.0 Hz, 1H), 4.02 (dd, J=7.2, 2.4 Hz, 1H), 3.83 (dd, J=12.0, 3.6 Hz, 1H), 3.74 (s, 2H), 3.64 (s, 1H), 3.45 (dd, J=7.2, 3.6 Hz, 0.33H), 3.41 (dd, J=7.2, 3.6 Hz, 066H), 2.27 (dd, J=3.6, 2.4 Hz, 0.66H), 2.19 (dd, J=3.6, 2.4 Hz, 0.33H). ¹³C NMR (50 MHz, CDCl₃) Mixture of rotamers δ170.1 and 169.1 (s), 156.2 (s), 135.7 (s), 128.2-127.2 (d, 5C), 80.9 (s), 67.7 (t), 65.9 and 65.4 (t), 57.6 (d), 54.9 (d) 52.5 (q), 35.0 (d), 28.4 and 28.1 (q). MS (m/z) 335 (M⁺-t-Bu, 2), 318 (0.3), 291 (1), 200 (16), 91 (100).

### Example 48

### Synthesis of methyl (1R,3R,4S,6S,7S)-3-Methyl-2-oxa-5-aza-bicyclo[4.1.0]heptane-4,5,7-tricarboxylic acid 5-benzyl ester 7-tert-butyl ester 4-methyl ester [structure XIII where X═CO, R₁═CH₃, R₂═COOMe, R₃═PhCH₂O—, R₁₃═H, R₁₄═COOt-Bu]

To a solution of compound VIIIa (where X═CO, R₁═CH₃, R₂═COOMe, R₃═PhCH₂O—) (450 mg, 1.54 mmol) in dry CH₂Cl₂ (4 mL) cooled in an ice-salt bath were added Cu(OTf)₂ (14 mg, 0.038 mmol), (S,S)-2,2′-Isopropylidene-bis(4-tert-butyl-2-oxazoline) (11 mg, 0.038 mmol) and phenylhydrazine (3.0 μL, 0.031 mmol). After 30 min, a 1.2 M solution of tert-butyl-diazoacetate (5 eq.) in dry CH₂Cl₂ was added during 6 h. During the addition the volume was maintained constant, expelling CH₂Cl₂ by passing nitrogen through the flask. The reaction was then gently warmed to room temperature and stirred 16 h. The mixture was concentrated under reduced pressure and the residue was purified by flash column chromatography (Hexanes-EtOAc 3:1) to yield 405 mg (65%) of XIII (where X═CO, R₁═CH₃, R₂═COOMe, R₃═PhCH₂O—, R₁₃═H, R₁₄═COOt-Bu). [α]²⁴_{D} 34.6 (c=1.9, CHCl₃). ¹H NMR (400 MHz, CDCl₃) 3:1 Mixture of rotamers δ7.34-7.24 (m, 5H), 5.30-5.05 (m, 2H), 4.34 (q, J=6.8 Hz, 0.75H), 4.27 (q, J=6.8 Hz, 0.25H), 4.08 (s, 0.75H), 4.03 (s, 0.25H), 3.80-3.75 (2 dd, J=7.2, 3.2 Hz, 1H), 3.68 (s, 2.25H), 3.58 (s, 0.75H), 3.54 (dd, J=7.2, 3.2 Hz, 0.25H), 3.48 3.54 (dd, J=7.2, 3.2 Hz, 0.75H), 2.27 (t, J=3.2 Hz, 0.75H), 2.17 (t, J=3.2 Hz, 0.25H), 1.45-1.41 (m, 5.25H), 1.35 (s, 6.75H). ¹³C NMR (50 MHz, CDCl₃) Mixture of rotamers δ170.5 (s), 169.4 (s), 156.8 (s), 135.7 (s), 128.3-127.2 (d, 5C), 80.7 (s), 70.1 and 69.6 (d), 67.6 (t), 58.8 and 58.4 (d), 52.5 (q), 34.9 (d), 27.9 and 27.6 (q, 3C), 17.8 (q). MS m/z 405 (M+, 0.1), 305 (2), 260 (9), 214 (51), 91 (100).

### Example 49

### Synthesis of (1R,4S,6S,7S)-2-oxa-5-aza-bicyclo[4.1.0]heptane-4,5,7-tricarboxylic acid 5-benzyl ester 4-methyl ester [structure XIII where X═CO, R₁═H, R₂═COOMe, R₃═PhCH₂O—, R₁₃═H, R₁₄═COOH]

Compound XIII (where X═CO, R₁═H, R₂═COOMe, R₃═—OCH₂Ph, R₁₃═H, R₁₄═COOt-Bu) (240 mg, 0.61 mmol) was dissolved in CH₂Cl₂ (2.8 mL) and TIS (125 μL, 0.61 mmol) and TFA (1.2 mL) were added sequentially. The mixture was stirred 50 minutes at room temperature and then the solvents were removed under reduced pressure. The crude product obtained was redissolved in 5% Na₂CO₃ (20 mL) and the solution was extracted with Et₂O (2×10 mL). The acqueous phase was acidified at pH 1-2 with concentrated HCl 37% and extracted with CH₂Cl₂ (4×10 mL). The dichloromethane extracts were combined, dried over Na₂SO₄ and concentrated under reduced pressure to obtain compound XIII (where X═CO, R₁═H, R₂═COOMe, R₃═PhCH₂O—, R₁₃═H, R₁₄═COOH) (174 mg, 85%). [α]²⁶_{D} 5.8 (c=1, CHCl₃). ¹H NMR (400 MHz, CDCl₃) δ: 2 Mixture of rotamers δ7.36-7.28 (m, 5H), 5.27-5.10 (m, 2H), 4.25 (dd, J=16.0, 2.8 Hz, 1H), 4.17-4.05 (m, 2H), 3.85-3.78 (m, 1H), 3.74 (s, 1.8H), 3.61 (s, 1.2H), 3.59-3.55 (m, 1H), 2.39 (s, 0.6H), 2.29 (s, 0.4H). ¹³C NMR (50 MHz, CDCl₃) Mixture of rotamers δ175.6 and 175.0 (s), 170.5 and 170.2 (s), 156.3 and 155.8 (s), 135.8 and 135.4 (s), 128.4-127.3 (d, 5C), 68.0 and 67.8 (t), 65.9 and 65.5 (t), 60.6 (t), 58.4 and 58.2(d), 55.4 and 54.9 (d), 52.6 (q), 36.0 and 35.8 (d), 27.4 (d). MS m/z 335 (M+, 0.2), 290 (0.3), 246 (6), 232 (3), 200 (11), 91 (100). Anal. calcd for C₁₆H₁₇NO₇: C, 57.31; H, 5.11; N, 4.18. Found: C, 57.36; H, 5.21; H, 4.19.

### Example 50

### Synthesis of (1R,4S,6S,7R)-7-Hydroxymethyl-2-oxa-5-aza-bicyclo[4.1.0]heptane-4,5-dicarboxylic acid 5-benzyl ester 4-methyl ester [structure XIII where X═CO, R₁═H, R₂═COOMe, R₃═PhCH₂O—, R₁₃═H, R₁₄═—CH₂OH]

N-Methylmorpholine (52 μL, 0.47 mmol) and isobutyl chloroformiate (61 μL, 0.45 mmol) were added, at 0° C., to a solution of compound XIII (where X═CO, R₁═H, R₂═COOMe, R₃═PhCH₂O—, R₁₃═H, R₁₄═—COON) (144 mg, 0.43 mmol) in dry THF (4 mL). After 25 minutes, the white suspension was added dropwise at −78° C. to a suspension of NaBH₄ (32 mg, 0.86 mmol) in THF/MeOH 3:1 (4 mL). After 30 minutes at −78° C. a second portion of NaBH₄ (32 mg, 0.86 mmol) was added and the mixture was stirred another 30 minutes at −78° C. and then was gently warmed to −40° C., until all the mixed anidride was consumed (TLC monitoring). The reaction was quenched with 10% AcOH/H₂O (2 mL), diluted with H₂O (8 mL), and extracted with AcOEt (3×15 mL). The combined organic extracts were washed with brine, dried over Na₂SO₄ and concentrated under reduced pressure to a residue which was purified by flash column chromatography (EtOAc—Hexanes 3:1 and then EtOAc) to yield alcohol XIII (where X═CO, R₁═H, R₂═COOMe, R₃═PhCH₂O—, R₁₃═H, R₁₄═—CH₂OH) (112 mg, 81%). [α]²⁵_{D} 74.6 (c=1.1, CHCl₃). ¹H NMR (400 MHz, CDCl₃) Approximately 1:1 mixture of rotamers δ7.40-7.29 (m, 5H), 5.29-5.10 (m, 2H), 4.10 (d, J=3.2 Hz, 0.5H), 4.02 (d, J=3.2 Hz, 0.5H), 3.83-3.72 (m, 2H), 3.74 (s, 1.5H), 3.64-3.59 (m, 1H), 3.58 (s, 1.5H), 3.26-3.19 (m, 1H), 2.76-2.70 (m, 1H), 1.82-1.80 (m, 1H). ¹³C NMR (50 MHz, CDCl₃) Mixture of rotamers δ, 170.8 and 170.5 (s), 156.6 (s), 135.8 and 135.6 (s), 128.4-127.8 (d, 5C), 67.8 (t), 66.1 and 65.8 (t), 61.1 and 60.8 (t), 56.2 and 55.7 (d), 55.4 and 54.9 (d), 52.5 (q), 30.3 and 30.0 (d), 28.6 and 28.2 (d). MS (m/z) 303 (M⁺-OH, 1), 290 (4), 218 (2), 200 (2), 91 (100). Anal. calcd for C₁₆H₁₉NO₆: C, 59.81; H, 5.96; N, 4.36. Found: C, 59.98; H, 5.71; N, 5.02.

1. Cyclic compound of general formula (I) 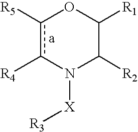
wherein:
a is a single or double bond;
X is chosen in the group consisting of “bond”, CO, SO₂, CS;
R₁ is chosen in the group consisting of H, C₁₋₈alkyl, C₂₋₈alkenyl, C₂₋₈alkynyl, C₁₋₈cycloalkyl, aryl, heterocycle, arylC₁₋₈alkyl; heterocycloC₁₋₈alkyl; —CH₂OR, RO—C₁₋₈alkyl, —CH₂NRR′, RR′N—C₁₋₈alkyl, RR′N-aryl, RO-aryl, R(O)C-aryl, RO(O)C-aryl, RR′N(O)C-aryl;
R₂ is chosen in the group consisting of α-amino acid side chain, —CO₂alkyl, —CH₂Oalkyl, —CH₂Oaryl, —CH₂OPg, —C(O)NRR′, —C(O)NHR′;
R₁ and R₂ can form a cycle;
R₃ is chosen in the group consisting of H, C₁₋₈alkyl, C₂₋₈alkenyl, C₂₋₈alkynyl, cycloalkyl, aryl, heterocicle, arylC₁₋₈alkyl; heterocycloC₁₋₈alkyl; RR′N—C₁₋₈alkyl, RR′N-aryl, RO-aryl, R(O)C-aryl, RO(O)C-aryl, RR′N(O)C-aryl, —CH(aryl)CO₂R, —CH(hetocycle)CO₂R, —CH(alkenyl)CO₂R, when X is bond;
is chosen in the group consisting of C₁₋₈alkyl, C₂₋₈alkenyl, C₂₋₈alkynyl, cycloalkyl, aryl, heterocycle, arylC₁₋₈alkyl; heterocycloC₁₋₈alkyl; RR′N—C₁₋₈alkyl, RR′N-aryl, RO-aryl, R(O)C-aryl, RO(O)C-aryl, RR′N(O)C-aryl, —CH(α-amino acid side chain)NHR₆, —OCH₂Ph, —OCH₂fluorenyl, —OCH₂-aryl, arylalkyloxy, —NHCH₂Ph, —NRR′ when X is other than bond;
can form a cycle with R₂ or R₄
R₄ is chosen in the group consisting of H, α-amino acid side chain;
or is C₁₋₈alkylidene when forms a cycle with R₅ and a is single bond;
R₅ is H when a is a double bond;
or is —OR when a is a single bond;
or can form a cycle with R₄ when R₄ is C₁₋₈alkylidene and a is single bond;
R₆ is chosen in the group consisting of —CO₂alkyl, —CO₂aryl, —SO₂aryl, —SO₂alkyl, a protecting group for amines, a peptide chain;
R is chosen in the group consisting of H, C₁₋₈alkyl, allyl, C₂₋₈alkenyl, acetyl, —C(O)—C₁₋₈alkyl; acryloyl, —C(O)—C₂₋₈alkenyl, aryl, benzyl, arylC₁₋₈alkyl, Pg;
R′ is chosen in the group consisting of C₁₋₈alkyl, benzyl, arylalkyl, allyl, C₂₋₈alkenyl, propargyl, C₂₋₈alkinyl cycloalkyl, acryloyl, —OR, —CO—C₂₋₈alkenyl, —CO—CH(α-amino acid side chain)NHR₆;
R and R′ together with N can form a cycle;
Pg is a protecting group for alcohols, amines or carboxylic acids;
the above said alkyl-, alkylidene, alkenyl-, alkynyl-, cycloalkyl-, aryl- and heterocyclic groups being able to be variably substituted.

2. Cyclic compound according to claim 1 of formula (I) wherein a, X, R₃, R₅, R₆, Pg, R and R′ are as defined in claim 1 and where:
i. when R₄ is α-amino acid side chain or is C₁₋₈alkylidene and forms a cycle with R₅ then
R₁ is chosen in the group consisting of —CH₂OR, —CH₂NRR′;
R₂ is chosen in the group consisting of —CO₂alkyl, —CH₂Oalkyl, —CH₂Oaryl, —CH₂OPg, —C(O)NRR′, —C(O)NHR′;
R₁ and R₂ can form a cycle;
R₂ and R₃ can form a cycle;
R₃ and R₄ can form a cycle;
ii. when R₂ is chosen in the group consisting of —CO₂alkyl, —CH₂Oalkyl, —CH₂Oaryl, —CH₂OPg, —C(O)NRR′, —C(O)NHR′, α-amino acid side chain then
R₁ is chosen in the group consisting of H, C₁₋₈alkyl, aryl, C₁₋₈alkyl, C₂₋₈alkenyl, C₂₋₈alkynyl, C₁₋₈cycloalkyl, heterocycle, arylC₁₋₈alkyl; heterocycloC₁₋₈alkyl;
R₄ is H or is C₁₋₈alkylidene and forms a cycle with R₅ when a is single bond;
and wherein the above said alkyl-, alkylidene, alkenyl-, alkynyl-, cycloalkyl-, aryl- and heterocyclic groups being able to be variably substituted.

3. Cyclic compounds according to claim 2 wherein R₄ is α-amino acid side chain and characterized by 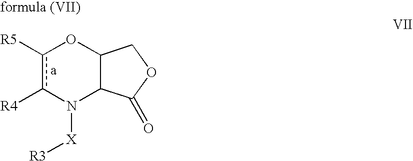
wherein:
a is a single or double bond;
X is chosen in the group consisting of “bond”, CO, SO₂;
R₃ and R₄ can form a cycle;
R₃ and R₅ are as defined in claim 1; 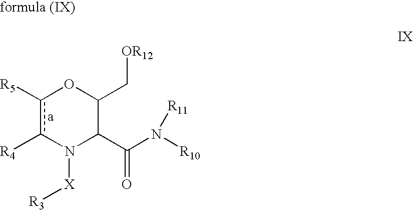
wherein:
a is a single or double bond;
X is chosen in the group consisting of CO, SO₂; bond if a is a single bond;
R₃ and R₄ can form a cycle;
R₁₀ and R₁₁ are independently chosen in the group consisting of H, C₁₋₈alkyl, C₂₋₈alkenyl, C₂₋₈alkynyl, C₁₋₈cycloalkyl, aryl, heterocycle, arylC₁₋₈alkyl; heterocycloC₁₋₈alkyl; —OR;
R₁₀ and R₁₁ can form a cycle;
R₁₂ is R;
R₃, R₅ and R are as defined in claim 1; 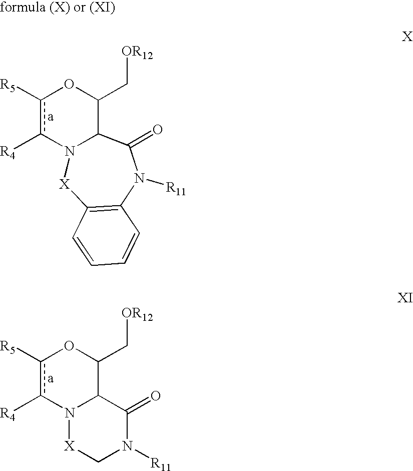
wherein:
a is a single or double bond
X is SO₂, CO
R₁₁ is chosen in the group consisting of H, C₁₋₈alkyl, C₂₋₈alkenyl, C₂₋₈alkynyl, C₁₋₈cycloalkyl, aryl, heterocycle, arylC₁₋₈alkyl; heterocycloC₁₋₈alkyl; —OR;
R₅ and R are as defined in claim 1; 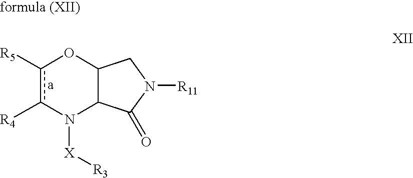
wherein:
a is a single or double bond;
X is SO₂, CO;
R11 is chosen in the group consisting of H, C₁₋₈alkyl, C₂₋₈alkenyl, C₂₋₈alkynyl, C₁₋₈cycloalkyl, aryl, heterocycle, arylC₁₋₈alkyl; heterocycloC₁₋₈alkyl; —OR;
R₃, R₅ and R are as defined in claim 1;
and wherein the above said alkyl-, alkylidene, alkenyl-, alkynyl-, cycloalkyl-, aryl- and heterocyclic groups being able to be variably substituted.

4. Cyclic compound according to claim 2 wherein R₂ is chosen in the group consisting of CO₂CH₃, CO₂CH₂CH₃, CO₂CH(CH₃)₂, CH₂OCH₂Ph, CH₂OPh, CH₂OH, CH₂OPg, α-amino acid side chain and characterised by 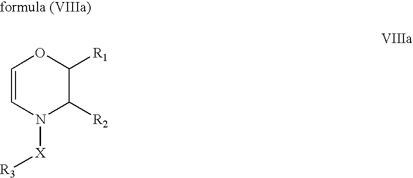
X is chosen in the group consisting of CO, SO₂;
R₁ is as defined in claim 2;
R₃ is as defined in claim 1; 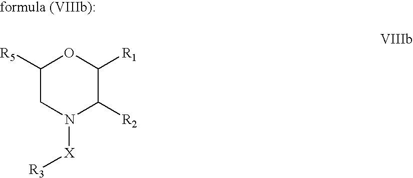
where:
X is chosen in the group consisting of “bond”, CO, SO₂;
R₁ is as defined in claim 2;
R₃ and R₅ are as defined in claim 1; 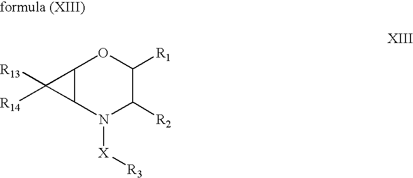
where:
X and R₃ are as defined in claim 1;
R₁ is as defined in claim 2;
R₁₃ is H and R₁₄ is H, COOMe, COOEt, COOtBu; CH₂OH, CH₂OPg, CH₂NHPg, NHPg;
R₁₃ and R₁₄ are both halogens.

5. Process for the preparation of compounds of formula (I) according to claim 1 which comprises the cyclization step of intermediate of formula (II) 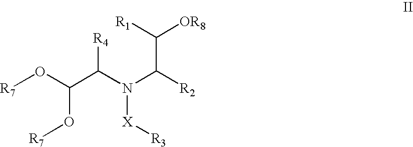
wherein R₁-R₄ and X are as defined in claim 2;
R₇ is chosen in the group consisting of methyl, ethyl or the two R₇ groups form a 1,3-dioxolane or 1,3-dioxane cycle;
R₈ is chosen in the group consisting of H, Pg;
R₁ and R₈ can form a 1,3-dioxalane cycle optionally substituted;
being Pg an acid-labile protecting group for alcohols.

6. Process according to claim 5 for the preparation of compound of formula (I) wherein a is single bond and wherein said cyclization step is performed by reaction in a polar protic solvent in the presence of acid catalysis.

7. Process according to claim 5 for the preparation of compound of formula (I) wherein a is double bond and wherein said cyclization step is performed by reaction in an apolar aprotic solvent in the presence of acid catalysis and in the presence of a hygroscopic agent

8. Process according to claim 6 for the preparation of compounds of formula (VII) according to claim 3 wherein a is single bond, wherein the intermediate to be cyclised is of formula (IIa) 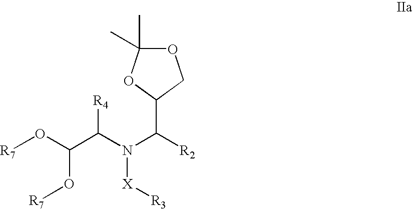
wherein:
X is chosen in the group consisting of “bond”, CO, SO₂;
R₂ is CO₂CH₃;
R₃ is as defined in claim 1;
R₄ is a α-amino acid side chain;
or the two R₇ groups form a 1,3-dioxalane or 1,3-dioxane cycle.

9. Process according to claim 6 for the preparation of compounds of formula (VIIIb) according to claim 4, wherein the intermediate to be cyclised is of formula (IIb) 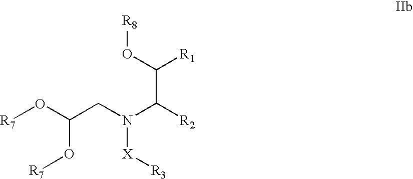
wherein:
X is chosen in the group consisting of CO, COO, CON, SO₂;
R₁ is as defined in claim 2;
R₂ is chosen between CO₂CH₃, CO₂CH₂CH₃, CH₂OCH₂Ph, CH₂OPh, CH₂OPg, α-amino acid side chain;
R₃ is as defined in claim 1;
R₇ is as defined in claim 5;
R₈ is defined in claim 5.

10. Process for the preparation of compounds of formulae (IX)-(XII) according to claim 3 comprising
the reaction of a compound of formula (VII) according to claim 3 with an amine or hydroxylamine.

11. Process for the preparation of intermediate of formula (II) according to claim 5 comprising the condensation of a compound of formula (V) or (Va) 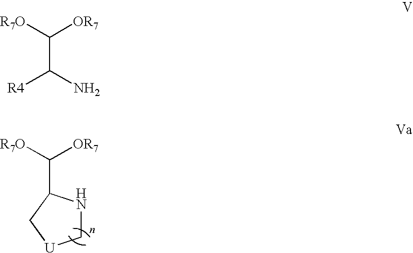
wherein
R₄ is an α-amino acid side chain;
R₇ is chosen in the group consisting of —C₁₋₈alkyl, —C₁₋₈cycloalkyl, aryl, Pg;
U is —CH₂—, CH(OPg)-, CH₂—CH(OPg)-, CH(OPg)-CH₂—, —CH(NPg)-, —O—, —S—, —N(Pg)-;
Pg is a protecting group for alcohols or amines;
n=1, 2;
with a compound of formula (VI) 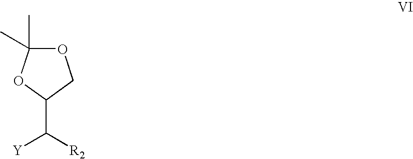
wherein
R₂ is chosen in the group consisting of —CO₂alkyl, —CH₂Oalkyl, —CH₂Oaryl, —CH(OPg;
Y is a leaving group.

12. Process for the preparation of intermediate of formula (II) according to claim 5 comprising the condensation of a compound of formula (III) 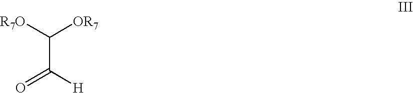
wherein R₇ is as defined in claim 5;
with a compound of formula (IV) 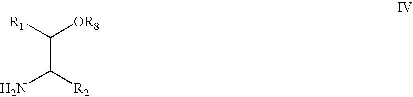
wherein
R₁ is as defined in claim 2;
R₂ is chosen in the group consisting of —CO₂alkyl, α-amino acid side chain;
R₈ is as defined in claim 5;

13. A combinatorial library comprising compounds of formula (I) according to claim 1.

14. A method for drug discovery screening wherein the combinatorial library according to claim 16 is used.

15. A method for the preparation of compounds for therapeutic use, said method wherein compounds of formula (I) according to claim 1 are used as intermediates.

16. A process according to claims 7 for the preparation of compounds of formula (VII) according to claim 3 wherein a is single bond, wherein the intermediate to be cyclised is of formula (IIa) 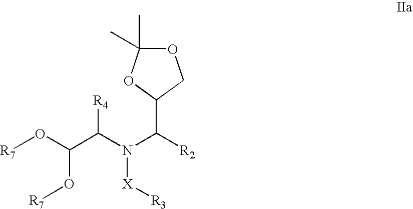
wherein:
X is chosen in the group consisting of “bond”, CO, SO₂;
R₂ is CO₂CH₃;
R₃ is as defined in claim 1;
R₄ is a α-amino acid side chain;
or the two R₇ groups form a 1,3-dioxalane or 1,3-dioxane cycle.

17. A process according to claims 7 for the preparation of compounds of formula (VIIIa) according to claim 4, wherein the intermediate to be cyclised is of formula (IIb) 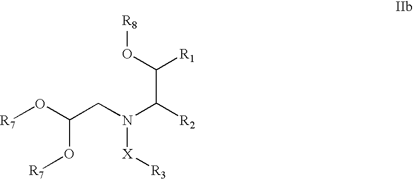
wherein:
X is chosen in the group consisting of CO, COO, CON, SO₂;
R₁ is as defined in claim 2;
R₂ is chosen between CO₂CH₃, CO₂CH₂CH₃, CH₂OCH₂Ph, CH₂OPh, CH₂OPg, α-amino acid side chain;
R₃ is as defined in claim 1;
R₇ is as defined in claim 5;
R₈ is defined in claim 5.

18. A process for the preparation of compounds of formula (XIII) according to claim 4 comprising the reaction of a compound of formula (VIIIa) according to claim 4 with a carbenoid species.
